(19) United States
(12) Patent Application Publication
(10) Pub. No.: US 2010/0028889 A1
(43) Pub. Date: Feb. 4, 2010

(54) COMPANION DIAGNOSTIC ASSAYS FOR CANCER THERAPY

(75) Inventors: Mark G. Anderson, Grayslake, IL (US); Paul E. Kroeger, Libertyville, IL (US); Saul H. Rosenberg, Grayslake, IL (US); Stephen K. Tahir, Fremont, CA (US); John A. Wass, Lake Forest, IL (US); Christin Tse, Libertyville, IL (US); Viswanath Devanarayan, Southerton, PA (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/490,558

(22) Filed: Jun. 24, 2009

Related U.S. Application Data
(63) Continuation-in-part of application No. 11/999,330, filed on Dec. 4, 2007.
(60) Provisional application No. 60/872,668, filed on Dec. 4, 2006.

(51) Int. Cl. C12Q 1/68; G06F 7/00

(52) U.S. Cl. 435/6; 707/104.1; 707/E17.044

(57) ABSTRACT

Methods for identifying cancer patients eligible to receive Bcl-2 family inhibitor therapy and for monitoring patient response to Bcl-2 family inhibitor therapy comprise assessment of the expression levels of the biomarker combinations set out in TABLES 1, 2, 3, 4, 5, 6, 7, 8, 9, 10, 11, 12, 13, 14, 15, 16, 17, 18, 19 or 20 in a patient tissue sample. The methods of the invention allow more effective identification of patients to receive Bcl-2 family inhibitor therapy and of determination of patient response to the therapy.

### RELATED APPLICATION INFORMATION

This application is a continuation-in-part of U.S. application Ser. No. 11/999,330 filed on Dec. 4, 2007, which claims the benefit of U.S. application Ser. No. 60/872,668 filed on Dec. 4, 2006, the contents of each of which are herein incorporated by reference.

### FIELD OF THE INVENTION

This invention relates to diagnostic assays useful in classification of patients for selection of cancer therapy, and in particular relates to measurements of expression signatures, particularly biomarker combinations, where the signatures correlate with responsiveness to cancer therapy and particularly Bcl-2-family antagonist therapy. Additionally, methods of the present invention, and particularly the biomarker combinations, are useful in the identification of patients eligible to receive Bcl-2-family antagonist therapy and that permit monitoring of patient response to such therapy.

### BACKGROUND OF THE INVENTION

Genetic heterogeneity of cancer is a factor complicating the development of efficacious cancer drugs. Cancers that are considered to be a single disease entity according to classical histopathological classification often reveal multiple genomic subtypes when subjected to molecular profiling. In some cases, molecular classification proved to be more accurate than the classical pathology. The efficacy of targeted cancer drugs may correlate with the presence of a genomic feature, such as a gene amplification, Cobleigh, M. A., et al., “Multinational study of the efficacy and safety of humanized anti-HER2 monoclonal antibody in women who have HER2-overexpressing metastatic breast cancer that has progressed after chemotherapy for metastatic disease”, J. Clin. Oncol., 17: 2639-2648, 1999; or a mutation, Lynch, T. J., et al., “Activating mutations in the epidermal growth factor receptor underlying responsiveness of non-small-cell lung cancer to gefitinib”, N. Engl. J. Med., 350: 2129-2139, 2004. For Her-2 in breast cancer, it has been demonstrated that detection of gene amplification provides superior prognostic and treatment selection information as compared with the detection by immunohistochemistry (IHC) of the protein overexpression, Pauletti, G., et al., “Assessment of Methods for Tissue-Based Detection of the HER-2/neu Alteration in Human Breast Cancer: A Direct Comparison of Fluorescence In Situ Hybridization and Immunohistochemistry”, J. Clin. Oncol., 18: 3651-3664, 2000. Cell line expression pattern data has specifically been shown to be predictive for patient sensitivity to chemotherapeutics Potti A., et al., Nat. Med. 2006 Epub ahead of print, PMID: 17057710. A need therefore exists for genomic classification markers that may improve the response rate of patients to targeted cancer therapy.

Targeted cancer therapy research has been reported against members of the Bcl-2 protein family, which are central regulators of programmed cell death. The Bcl-2 family members that inhibit apoptosis are overexpressed in cancers and contribute to tumorigenesis. Bcl-2 expression has been strongly correlated with resistance to cancer therapy and decreased survival.

A compound called ABT-737 is a small-molecule inhibitor of the Bcl-2 family members Bcl-2, Bcl-XL, and Bcl-w, and has been shown to induce regression of solid tumors, Oltersdorf, T., “An inhibitor of Bcl-2 family proteins induces regression of solid tumors”, Nature, 435: 677-681, 2005. ABT-737 has been tested against a diverse panel of human cancer cell lines and has displayed selective potency against SCLC and lymphoma cell lines, Ibid. ABT-737's chemical structure is provided by Oltersdorf et al. at p. 679.

Because of the potential therapeutic use of inhibitors for Bcl-2 family members, companion diagnostic assays that would identify patients eligible to receive Bcl-2 family inhibitor therapy are needed. Additionally, there is a clear need to support this therapy with diagnostic assays using biomarkers that would facilitate monitoring the efficacy of Bcl-2 family inhibition therapy.

### SUMMARY OF THE INVENTION

The present invention relates to the identification and use of gene expression patterns (or profiles or signatures), which are clinically relevant to cancer therapy. In particular, the identities of genes that are correlated with the identification, treatment and monitoring of patients for cancer treatment and particularly Bcl-2 family antagonist therapy.

The invention provides companion diagnostic assays for classification of patients for cancer treatment which comprise assessment in a patient tissue sample the levels of biomarker combinations set out in TABLES 1, 2, 3, 4, 5, 6, 7, 8, 9, 10, 11, 12, 13, 14, 15, 16, 17, 18, 19 or 20. The inventive assays include assay methods for identifying patients eligible to receive Bcl-2 family antagonist therapy and for monitoring patient response to such therapy. The invention methods comprise assessment of the biomarkers in blood, urine, or other body fluid samples by immunoassay, proteomic assay or nucleic acid hybridization or amplification assays, and in tissue or other cellular body samples by immunohistochemistry or in situ hybridization assays.

Gene expression patterns of the invention are identified as described below. Generally a large sampling of the gene expression profile of a sample is obtained through quantifying the expression levels of mRNA corresponding to many genes identified in the biomarker combinations. The profile, or combination set is then analyzed to identify genes, the expression of which are positively correlated with the identification and monitoring of patients eligible of cancer treatment and particularly Bcl-2 family antagonist therapy.

In a preferred embodiment, the invention comprises a method for identifying a patient as eligible to receive cancer therapy, and preferably Bcl-2 family inhibitor therapy comprising: (a) providing a peripheral blood sample from a patient; (b) determining expression levels in the peripheral blood sample of biomarker combinations set out in TABLES 1, 2, 3, 4, 5, 6, 7, 8, 9, 10, 11, 12, 13, 14, 15, 16, 17, 18, 19 or 20; and (c) classifying the expression levels relative to normal peripheral blood levels of biomarker combinations set out in TABLES 1, 2, 3, 4, 5, 6, 7, 8, 9, 10, 11, 12, 13, 14, 15, 16, 17, 18, 19 or 20; and (d) identifying the patient as eligible for cancer therapy and preferably Bcl-2 family inhibitor therapy where the patient's blood sample is classified as having elevated expression levels of biomarker combinations set out in TABLES 1, 2, 3, 4, 5, 6, 7, 8, 9, 10, 11, 12, 13, 14, 15, 16, 17, 18, 19 or 20. In this embodiment, levels in the peripheral blood sample is preferably determined by a polymerase chain reaction (PCR) assay, for example, or performed on a lung cancer tumor biopsy sample.

In a preferred embodiment, the invention comprises a method for identifying a patient as eligible for cancer therapy, most preferably Bcl-2 family inhibitor therapy, comprising: (a) providing a tissue or cellular sample from a patient; (b) contacting the tissue or cellular sample with a labeled antibody or protein capable of binding to the biomarker combinations set out in TABLES 1, 2, 3, 4, 5, 6, 7, 8, 9, 10, 11, 12, 13, 14, 15, 16, 17, 18, 19 or 20; (c) classifying the expression levels relative to normal tissue or cellular level of the biomarker combinations set out in TABLES 1, 2, 3, 4, 5, 6, 7, 8, 9, 10, 11, 12, 13, 14, 15, 16, 17, 18, 19 or 20; and (d) identifying the patient as eligible for cancer therapy and most preferably Bcl-2 family therapy where the patient's sample is classified as having differential levels of members of the biomarker combinations set out in TABLES 1, 2, 3, 4, 5, 6, 7, 8, 9, 10, 11, 12, 13, 14, 15, 16, 17, 18, 19 or 20.

The invention has significant capability to provide improved stratification of patients for cancer therapy, and in particular for Bcl-2 family inhibitor therapy. The assessment of these biomarkers with the invention also allows tracking of individual patient response to the therapy. The inventive assays have particular utility for classification of small cell lung carcinoma (SCLC) and leukemia/lymphoma patients.

The invention also comprises a preferred method for monitoring a patient being treated for cancer and preferably with Bcl-2 family inhibitor therapy comprising: (a) providing a peripheral blood sample from a patient; (b) measuring expression levels in the peripheral blood sample of the biomarker combinations set out in TABLES 1, 2, 3, 4, 5, 6, 7, 8, 9, 10, 11, 12, 13, 14, 15, 16, 17, 18, 19 or 20; and (c) determining the expression level relative to a patient baseline blood level of the biomarker combinations set out in TABLES 1, 2, 3, 4, 5, 6, 7, 8, 9, 10, 11, 12, 13, 14, 15, 16, 17, 18, 19 or 20.

The invention also comprises a reagent kit for an assay for levels of the RNA from the biomarker combinations set out in TABLES 1, 2, 3, 4, 5, 6, 7, 8, 9, 10, 11, 12, 13, 14, 15, 16, 17, 18, 19 or 20, as well as a reagent kit for levels if at least one RNA from the biomarker combinations set out in TABLES 1, 2, 3, 4, 5, 6, 7, 8, 9, 10, 11, 12, 13, 14, 15, 16, 17, 18, 19 or 20. The invention has significant capability to provide improved stratification of patients for cancer therapy, and in particular for Bcl-2 family inhibitor therapy. The assessment of these biomarkers with the invention also allows tracking of individual patient response to the therapy. The inventive assays have particular utility for classification of SCLC and lymphoma patients.

### BRIEF DESCRIPTION OF THE DRAWINGS

FIG. 1 shows the expression profile for the biomarker combination groups that differentiate line sensitive and resistant to ABT-737 for small cell lung carcinoma cells (FIG. 1-A) and leukemia/lymphoma cells (FIG. 1-B).

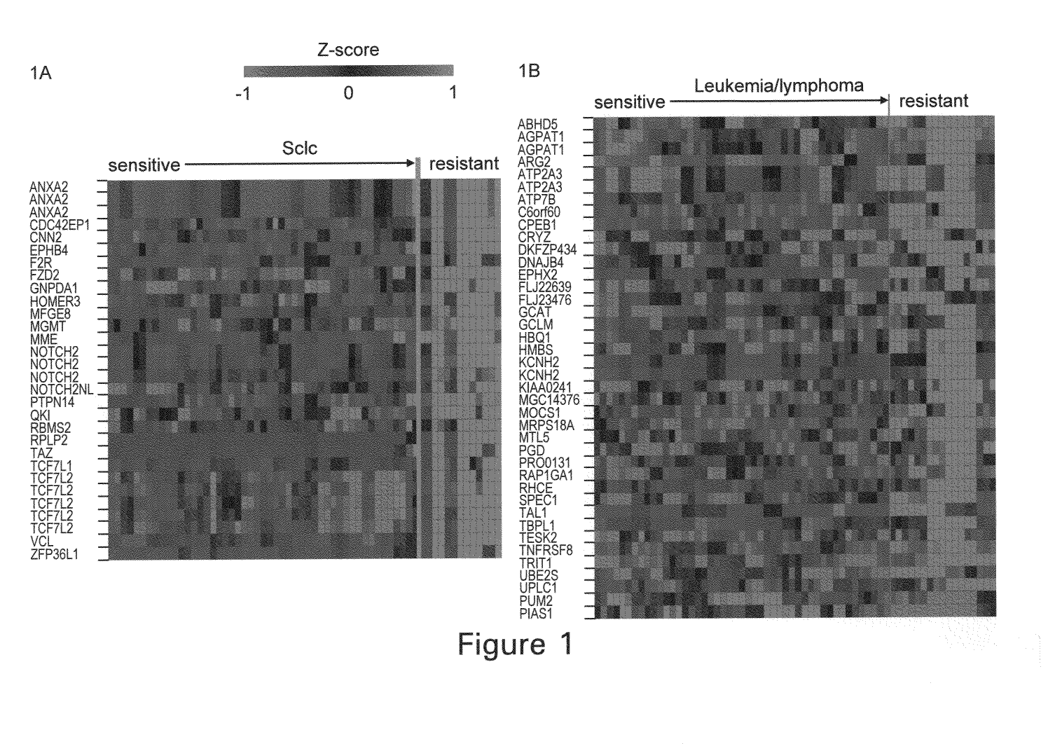

FIG. 2 shows the expression profile for the biomarker combination groups that differentiate line sensitive and resistant to ABT-263 for small cell lung carcinoma cells (FIG. 2-A) and leukemia/lymphoma cells (FIG. 2-B).

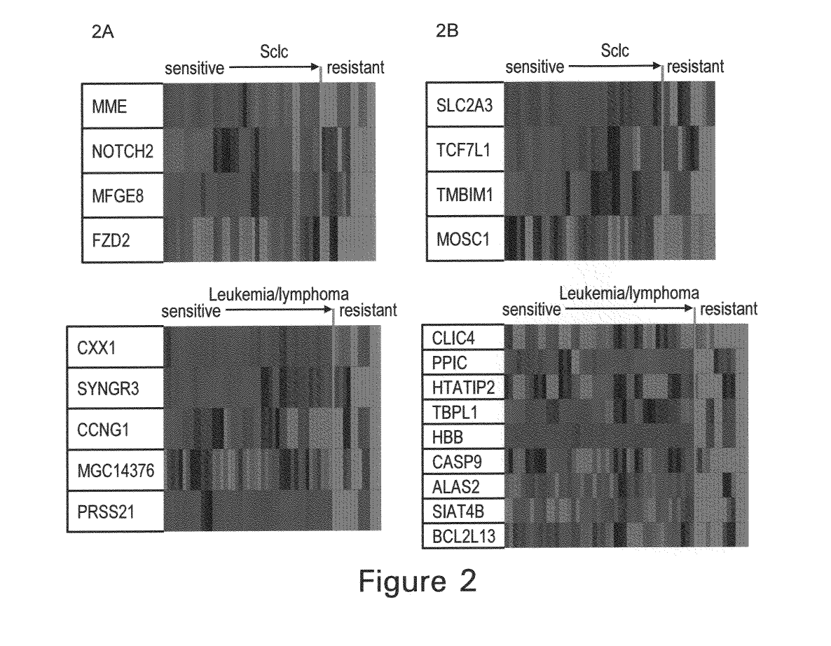

FIG. 3 shows EC₅₀ values plotted versus rank pursuant to Example 2. Specifically, calculated EC₅₀ values (in μM) were plotted based on rank, and a curve was fit using a fitting spline with lambda equal to 1 using JMP software (Version 6.0, SAD, Cary, N.C.). FIG. 3A shows SCLC cell lines. FIG. 3B shows leukemia/lymphoma cell lines.

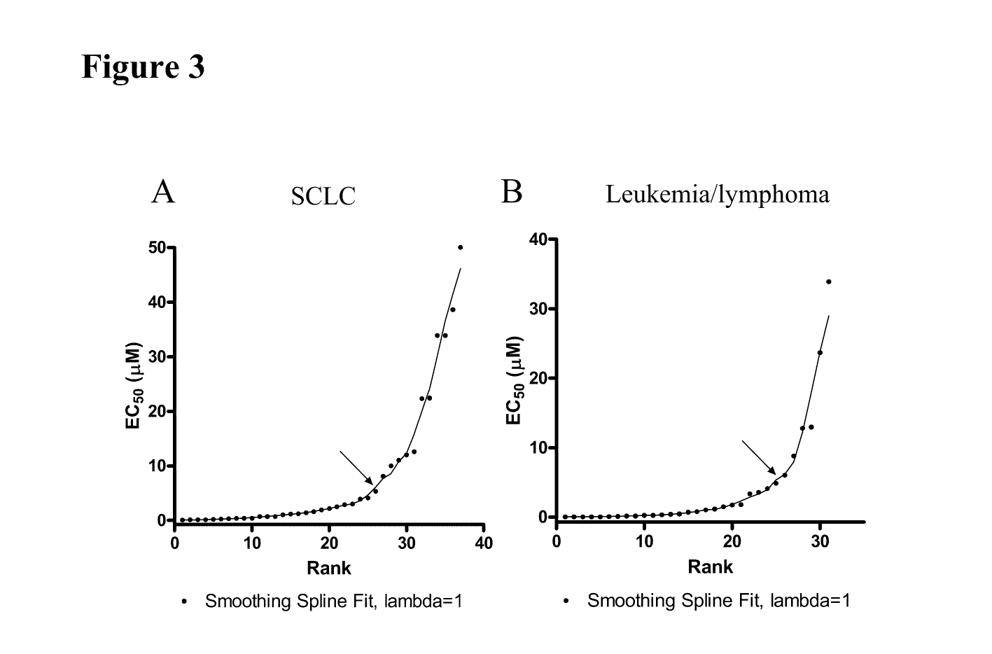

FIG. 4 shows a heat map of genes in the SCLC predictor set described in Example 2. The intensity values from the relevant probe sets for the training (used to identify signature sets) and test (used to test the identified signatures) cell lines were imported into Spotfire® software (available from TIBCO®), normalized together, and displayed using green for low expression and red for high expression as indicated in the color bar, with data from each microarray shown in the order of the corresponding EC₅₀ value (increasing from left to right). Expression values for the cell lines in the training set for the genes in predictor set 1 are shown in FIG. 4A, while expression values for the cell lines in the training set for the genes in predictor set 2 are shown in FIG. 4B. Set 1 and set 2 were combined, and a heat map of the best performing subset (FZD2, SLC2A3, and TMBIM1) is shown in FIG. 4C for the test cell lines.

FIG. 5 shows a heat map of genes in the leukemia/lymphoma predictor set as described in Example 2. The intensity values are displayed as described in FIG. 4 for the leukemia/lymphoma predictor set 1 (panel FIG. 5A) and predictor set 2 (panel FIG. 5B) for the training cell lines, and for the optimally performing combination set, C17orf91, CCNG1, PRSS21, and CASP9 (panel FIG. 5C), for the test cell lines.

FIG. 6 shows a heat map of expression of predictor set genes in primary SCLC tumors and normal lung tissue as described in Example 2. The intensity values are displayed as described in FIG. 4 for the SCLC predictor sets (panel FIG. 6A), and the leukemia/lymphoma sets (panel FIG. 6B) using the data from 8 SCLC tumor samples, and normal adjacent tissue from 6 of those tumors.

### DETAILED DESCRIPTION OF THE INVENTION

I. General

The invention is based on the discovery by Applicants of gene and gene signature groups in small cell lung cancer cell (SCLC) lines and leukemia/lymphoma (LL) cell lines that correlate to therapy resistance and sensitivity. In particular, Applicants correlated differential expression levels of novel biomarker combinations, which correlate to the sensitivity and resistance to a Bcl-2 family inhibitor.

As used herein, a “Bc1-2 family inhibitor” refers to a therapeutic compound of any type, including small molecule-, antibody-, antisense-, small interfering RNA-, or microRNA-based compounds, that binds to at least one of Bcl-2, Bcl-XL, and Bcl-w, and antagonizes the activity of the Bcl-2 family related nucleic acid or protein. The inventive methods are useful with any known or hereafter developed Bcl-2 family inhibitor. On example of a Bc1-2 family inhibitor is ABT-737, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide, which binds to each of Bcl-2, Bcl-XL, and Bcl-w. Another example of a Bcl-2 family inhibitor is ABT-263, N-(4-(4-((2-(4-chlorophenyl)-5,5-dimethyl- 1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide. The chemical structure of ABT-263 is:

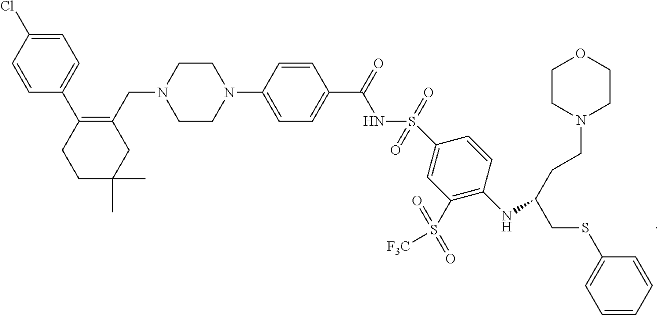

Other examples of Bcl-2 family related compounds useful in the present invention can be found in International Publication Numbers WO 05/049593 and WO 05/049594, both published on Jun. 2, 2005, incorporated by reference herein in its entirety.

The assays of the invention have potential use with targeted cancer therapy. In particular, the inventive assays are useful with therapy selection for small cell lung cancer and leukemia/lymphoma patients, such as therapy with Bcl-2 family inhibitors. Other examples of such cancers include solid tissue epithelial cancers, e.g., prostate, ovarian and esophageal cancer. The inventive assays are performed on a patient tissue sample of any type or on a derivative thereof, including peripheral blood, tumor or suspected tumor tissues (including fresh frozen and fixed or paraffin embedded tissue), cell isolates such as circulating epithelial cells separated, circulating tumor cell or identified in a blood sample, lymph node tissue, bone marrow and fine needle aspirates.

As used herein, Bcl-2 (official symbol BCL2) means the human B-cell CLL/lymphoma 2 gene; Bcl-x1 (official symbol BCL2L1) means the human BCL2-like 1 gene; Bcl-w (official symbol BCL2L2) means the human BCL2-like 2 gene.

As used herein, ANXA2 (official symbol ANXA2) annexin A2; CDC42EP1 (official symbol CDC42EP1); CDC42 (official symbol CDC42) effector protein (Rho GTPase binding 1); CNN2 (official symbol CNN2) calponin 2; EPHB4 (official symbol EPHB4) EPH receptor B4; F2R (official symbol F2R) coagulation factor II (thrombin) receptor; FZD2 (official symbol FZD2) frizzled homolog 2 (Drosophila); GNPDA1 (official symbol GNPDA1) glucosamine-6-phosphate deaminase 1; HOMER3 (official symbol HOMER3) homer homolog 3 (Drosophila); MFGE8 (official symbol MFGE8) milk fat globule-EGF factor 8 protein; MGMT (official symbol MGMT) O-6-methylguanine-DNA methyltransferase; MME (official symbol MME) membrane metallo-endopeptidase (neutral endopeptidase, enkephalinase, CALLA, C); NOTCH2 (official symbol NOTCH2) Notch homolog 2 (Drosophila); PTPN14 (official symbol PTPN14) protein tyrosine phosphatase, non-receptor type 14; QKI (official symbol QKI) quaking homolog, KH domain RNA binding (mouse); RBMS2 (official symbol RBMS2) RNA binding motif, single stranded interacting protein 2; TCF7L1 (official symbol TCF7L1) transcription factor 7-like 1 (T-cell specific, HMG-box); TCF7L2 (official symbol TCF7L2) transcription factor 7-like 2 (T-cell specific, HMG-box); VCL (official symbol VCL) vinculin; VIM (official symbol VIM) vimentin; WWTR1 (official symbol WWTR1) WW domain containing transcription regulator 1; ZFP36L1 (official symbol ZFP36L1) zinc finger protein 36, C3H type-like 1; PGD (official symbol PGD) phosphogluconate dehydrogenase; UBE2S (official symbol UBE2S) ubiquitin-conjugating enzyme E2S; CRYZ (official symbol CRYZ) crystallin, zeta (quinone reductase); HMBS (official symbol HMBS) hydroxymethylbilane synthase; DNAJB4 (official symbol DNAJB4) DnaJ (Hsp40) homolog, subfamily B, member 4; RAP1GA1 (official symbol RAP1GA1) RAP1, GTPase activating protein 1; GCLM (official symbol GCLM) glutamate-cysteine ligase, modifier subunit; ARG2 (official symbol ARG2) arginase, type II; ATP7B (official symbol ATP7B) ATPase, Cu++ transporting, beta polypeptide (Wilson disease); GCAT (official symbol GCAT) glycine C-acetyltransferase (2-amino-3-ketobutyrate coenzyme A ligase); KCNH2 (official symbol KCNH2) potassium voltage-gated channel, subfamily H (eag-related), member 2; TESK2 (official symbol TESK2) testis-specific kinase 2; TAL1 (official symbol TAL1) T-cell acute lymphocytic leukemia 1; TNFRSF8 (official symbol TNFRSF8) tumor necrosis factor receptor superfamily, member 8; ATP2A3 (official symbol ATP2A3) ATPase, Ca++ transporting, ubiquitous; TBPL1 (official symbol TBPL1) TBP-like 1; EPHX2 (official symbol EPHX2) epoxide hydrolase 2, cytoplasmic; KCNH2 (official symbol KCNH2) potassium voltage-gated channel, subfamily H (eag-related), member 2; MOCS1 (official symbol MOCS1) molybdenum cofactor synthesis 1; KIAA0241 (official symbol KIAA0241) KIAA0241 protein; MGC14376 (official symbol MGC14376) hypothetical protein MGC14376; YOD1 (official symbol YOD1) YOD1 OTU deubiquinating enzyme 1 homolog ( yeast); AGPAT1 (official symbol AGPAT1) 1-acylglycerol-3-phosphate O-acyltransferase 1 (lysophosphatidic acid acyltransferase, alpha); RHCE (official symbol RHCE) Rhesus blood group, CcEe antigens; CDC42SE1 (official symbol CDC42SE1) CDC42 small effector 1; TRIT 1 (official symbol TRIT 1) tRNA isopentenyltransferase 1; YRDC (official symbol YRDC) ischemia/reperfusion inducible protein; ABHD5 (official symbol ABHD5) abhydrolase domain containing 5; DDEFL1 (official symbol DDEFL1) development and differentiation enhancing factor-like 1; CPEB 1 (official symbol CPEB 1) cytoplasmic polyadenylation element binding protein 1; CCDC21 (official symbol CCDC21) coiled-coil domain containing 21; MTL5 (official symbol MTL5) metallothionein-like 5, testis-specific (tesmin); C6orf60 (official symbol C6orf60) chromosome 6 open reading frame 60; FLJ22639 (official symbol FLJ22639) hypothetical protein FLJ22639; HBQ1 (official symbol HBQ1) hemoglobin, theta 1; MRPS 1 8A (official symbol MRPS 18A) mitochondrial ribosomal protein S18A; AGPAT1 (official symbol AGPAT1) 1-acylglycerol-3-phosphate O-acyltransferase 1 (lysophosphatidic acid acyltransferase, alpha); PIAS1 (official symbol PIAS1) protein inhibitor of activated STAT, 1; PUM2 (official symbol PUM2) pumilio homolog 2 (Drosophila); SLC2A3 (official symbol SLC2A3) solute carrier family 2 (facilitated glucose transporter), member 3; transcription factor 7-like 2 (T-cell specific, HMG-box); TMBIM1 (official symbol TMBIM1) transmembrane BAX inhibitor motif containing 1; MOSC1 (official symbol MOSC1) MOCO sulphurase C-terminal domain containing 1; CXX1 (official symbol CXX1) CAAX box 1; SYNGR3 (official symbol SYNGR3) synaptogyrin 3; CCNG1 (official symbol CCNG1) cyclin GI; MGC14376 (official symbol MGC14376) hypothetical protein MGC14376; PRSS21 (official symbol PRSS21) protease, serine, 21 (testisin); CASP9 (official symbol CASP9) caspase 9, apoptosis-related cysteine peptidase; ALAS2 (official symbol ALAS2) aminolevulinate, delta-, synthase 2 (sideroblastic/hypochromic anemia); ST3GAL2 (official symbol ST3GAL2) ST3 beta-galactoside alpha-2,3-sialyltransferase 2; BCL2L13 (official symbol BCL2L13) BCL2-like 13 (apoptosis facilitator); PPIC (official symbol PPIC) peptidylprolyl isomerase C (cyclophilin C); CLIC4 (official symbol) chloride intracellular channel 4; TBPL1 (official symbol TBPL1) TBP-like 1; HBB (official symbol HBB) hemoglobin, beta /// hemoglobin, beta; and HTATIP2 (official symbol HTATIP2) HIV-1 Tat interactive protein 2, 30 kDa.

As used herein, “consisting essentially of” refers to the maximum number of genes that are required for the use of a biomarker to improve stratification of patents for cancer therapy, and in particular Bcl-2 family inhibitor therapy. In one embodiment, a biomarker to improve stratification of patents for cancer therapy, and in particular Bcl-2 family inhibitor therapy consisting essentially of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all of the biomarkers of the invention. In another embodiment, a biomarker to improve stratification of patients for cancer therapy, and in particular Bcl-2 family inhibitor therapy consisting essentially of any one of the biomarkers in TABLE 1. In another embodiment, a biomarker to improve stratification of patients for cancer therapy, and in particular Bc1-2 family inhibitor therapy consisting essentially of any one of the biomarkers in TABLE 2. In another embodiment, a biomarker to improve stratification of patients for cancer therapy, and in particular Bcl-2 family inhibitor therapy consisting essentially of any one of the biomarkers in TABLE 3. In another embodiment, a biomarker to improve stratification of patients for cancer therapy, and in particular Bcl-2 family inhibitor therapy consisting essentially of any one of the biomarkers in TABLE 4. In another embodiment, a biomarker to improve stratification of patients for cancer therapy, and in particular Bcl-2 family inhibitor therapy consisting essentially of any one of the biomarkers in TABLE 5. In another embodiment, a biomarker to improve stratification of patients for cancer therapy, and in particular Bc1-2 family inhibitor therapy consisting essentially of any one of the biomarkers in TABLE 6. In another embodiment, a biomarker to improve stratification of patients for cancer therapy, and in particular Bcl-2 family inhibitor therapy consisting essentially of any one of the biomarkers in TABLE 7. In another embodiment, a biomarker to improve stratification of patients for cancer therapy, and in particular Bcl-2 family inhibitor therapy consisting essentially of any one of the biomarkers in TABLE 8. In another embodiment, a biomarker to improve stratification of patients for cancer therapy, and in particular Bcl-2 family inhibitor therapy consisting essentially of any one of the biomarkers in TABLE 9. In another embodiment, a biomarker to improve stratification of patients for cancer therapy, and in particular Bc1-2 family inhibitor therapy consisting essentially of any one of the biomarkers in TABLE 10. In another embodiment, a biomarker to improve stratification of patients for cancer therapy, and in particular Bcl-2 family inhibitor therapy consisting essentially of any one of the biomarkers in TABLE 11. In another embodiment, a biomarker to improve stratification of patients for cancer therapy, and in particular Bcl-2 family inhibitor therapy consisting essentially of any one of the biomarkers in TABLE 12. In another embodiment, a biomarker to improve stratification of patients for cancer therapy, and in particular Bc1-2 family inhibitor therapy consisting essentially of any one of the biomarkers in TABLE 13. In another embodiment, a biomarker to improve stratification of patients for cancer therapy, and in particular Bcl-2 family inhibitor therapy consisting essentially of any one of the biomarkers in TABLE 14. In another embodiment, a biomarker to improve stratification of patients for cancer therapy, and in particular Bcl-2 family inhibitor therapy consisting essentially of any one of the biomarkers in TABLE 15. In another embodiment, a biomarker to improve stratification of patients for cancer therapy, and in particular Bcl-2 family inhibitor therapy consisting essentially of any one of the biomarkers in TABLE 16. In another embodiment, a biomarker to improve stratification of patients for cancer therapy, and in particular Bc1-2 family inhibitor therapy consisting essentially of any one of the biomarkers in TABLE 17. In another embodiment, a biomarker to improve stratification of patients for cancer therapy, and in particular Bcl-2 family inhibitor therapy consisting essentially of any one of the biomarkers in TABLE 18. In another embodiment, a biomarker to improve stratification of patients for cancer therapy, and in particular Bcl-2 family inhibitor therapy consisting essentially of any one of the biomarkers in TABLE 19. In another embodiment, a biomarker to improve stratification of patients for cancer therapy, and in particular Bcl-2 family inhibitor therapy consisting essentially of any one of the biomarkers in TABLE 20.

The biomarker combinations set out in TABLES 1, 2, 3, 4, 5, 6, 7, 8, 9, 10, 11, 12, 13, 14, 15, 16, 17, 18, 19 or 20 may be used alone or in combination with each other.

As used herein, the term “differential expression” refers to a difference in the level of expression of the RNA of one or more biomarkers of the invention, as measured by the amount or level mRNA, and/or one or more spliced variants of mRNA of the biomarker in one sample as compared with the level of expression of the same one or more biomarkers of the invention in a second sample. “Differentially expressed” can also include a measurement of the protein encoded by the biomarker of the invention in a sample or population of samples as compared with the amount or level of protein expression in a second sample or population of samples. Differential expression can be determined as described herein and as would be understood by a person skilled in the art.

The term “gene” refers to a nucleic acid (e.g., DNA) sequence that comprises coding sequences necessary for the production of a polypeptide, RNA (e.g., including but not limited to, mRNA, tRNA and rRNA) or precursor (e.g., precursors). The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In particular, the term “gene” refers to the full-length nucleotide sequence. However, it is also intended that the term encompass fragments of the sequence, as well as other domains within the full-length nucleotide sequence. Furthermore, the terms “nucleotide sequence” or “polynucleotide sequence” encompasses DNA, cDNA, and RNA (e.g., mRNA) sequences.

As used herein, a “gene expression pattern” or “gene expression profile” or “gene signature” refers to the relative expression of genes correlated with the classification of patients for cancer therapy and particularly Bcl-2-family antagonist therapy, as well as the expression of genes correlation with the responsiveness and monitoring of patients undergoing cancer therapy and particularly Bcl-2-family inhibitor therapy. Moreover, the terms “gene expression pattern” or “gene expression profile” or “gene signature” indicate that combined pattern of the results of the analysis of the level of expression of two or more biomarkers of the invention including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all of the biomarkers of the invention. A gene expression pattern or gene expression profile or gene signature can result from the measurement of expression of the RNA and/or the protein expressed by the gene corresponding to the biomarkers of the invention. In the case of RNA it refers to the RNA transcripts transcribed from genes corresponding to the biomarker of the invention. In the case of protein it refers to proteins translated from the genes corresponding to the biomarker of the invention. For example, techniques to measure expression of the RNA products of the biomarkers of the invention includes, PCR based methods (including RT-PCR) and non PCR based methods as well as microarray analysis. To measure protein products of the biomarkers of the invention, techniques include western blotting and ELISA analysis.

Because the invention relies upon the identification of genes that are over expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations), which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. Preferably, the sequences are those of mRNA encoded by the genes, the corresponding cDNA to such mRNAs, and/or amplified versions of such sequences. In preferred embodiments of the invention, the polynucleotide probes are immobilized on an array, other solid support devices, or in individual spots that localize the probes.

In another embodiment of the invention, all or part of a disclosed sequence may be amplified and detected by methods such as the polymerase chain reaction (PCR) and variations thereof, such as, but not limited to, quantitative PCR (Q-PCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization.

Alternatively, and in yet another embodiment of the invention, gene expression may be determined by analysis of expressed protein in a cell sample of interest by use of one or more antibodies specific for one or more epitopes of individual gene products (proteins) in said cell sample. Such antibodies are preferably labeled to permit their easy detection after binding to the gene product.

As used herein, the term “in combination” when referring to therapeutic treatments refers to the use of more than one type of therapy (e.g., more than one prophylactic agent and/or therapeutic agent). The use of the term “in combination” does not restrict the order in which therapies (e.g., prophylactic and/or therapeutic agents) are administered to a subject. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject.

Moreover, Bcl-2 inhibitor family therapy may also be administered in combination with one or more than one additional therapeutic agents, wherein additional therapeutic agents include radiation or chemotherapeutic agents, wherein chemotherapeutic agents include, but are not limited to, carboplatin, cisplatin, cyclophosphamide, dacarbazine, dexamethasone, docetaxel, doxorubicin, etoposide, fludarabine, irinotecan, CHOP (C: Cytoxan® (cyclophosphamide); H: Adiamycin® (hydroxydoxorubicin); O: Vincristine (Oncovin®); P: prednisone), paclitaxel, rapamycin, Rituxin® (rituximab) and vincristine.

As used herein, the term “level of expression” when referring to RNA refers to the measurable quantity of a given nucleic acid as determined by hybridization or measurements such as real-time RT PCR, which includes use of both SYBR® green and TaqMan® technology and which corresponds in direct proportion with the extent to which the gene is expressed. The level of expression of a nucleic acid is determined by methods well known in the art. For microarray analysis, the level of expression is measured by hybridization analysis using labeled nucleic acids corresponding to RNA isolated from one or more individuals according to methods well known in the art. The label on the nucleic acid used for hybridization can be a luminescent label, an enzymatic label, a radioactive label, a chemical label or a physical label. Preferably, target nucleic acids are labeled with a fluorescent molecule. Preferred fluorescent labels include, but are not limited to: fluorescein, amino coumarin acetic acid, tetramethylrhodamine isothiocyanate (TRITC), Texas Red, Cyanine 3 (Cy3) and Cyanine 5 (Cy5).

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

A “microarray” refers to an ordered arrangement of hybridizable array elements, preferably polynucleotide probes, on a support.

As used herein, the term “official symbol” refers to EntrezGene database maintained by the United States National Center for Biotechnology Information.

As used herein, the term “predetermined level” refers generally at an assay cutoff value that is used to assess diagnostic results by comparing the assay results against the predetermined level, and where the predetermined level already that has been linked or associated with various clinical parameters (e.g., monitoring whether a subject being treated with a drug has achieved an efficacious blood level of the drug, monitoring the response of a subject receiving treatment for cancer with an anti-cancer drug, monitoring the response of a tumor in a subject receiving treatment for said tumor, etc.). The predetermined level may be either an absolute value or a value normalized by subtracting the value obtained from a patient prior to the initiation of therapy. An example of a predetermined level that can be used is a baseline level obtained from one or more subjects that may optionally be suffering from one or more diseases or conditions.

The term “support” refers to conventional supports such as beads, particles, dipsticks, fibers, filters, membranes and silane or silicate supports such as glass slides.

The invention comprises diagnostic assays performed on a patient tissue sample of any type or a derivate thereof, including peripheral blood, tumor or suspected tumor tissues (including fresh frozen and fixed or paraffin embedded tissue), cell isolates such as circulating epithelial cells separated or identified in a blood sample. Lymph node tissue, bone marrow and fine needle aspirates. Preferred tissue samples for use herein are peripheral blood, tumor or suspected tumor tissue and bone marrow.

II. Bcl-2 Family Inhibitor Biomarkers

Applicants identified novel biomarker combinations useful for stratifying and/or monitoring patient's response to cancer therapy and particularly to Bcl-2 family inhibitor therapy.

The invention comprises assessment in a patient tissue sample of levels of the genes in the biomarker sets, by measurement of these genes at their expressed protein level or translated messenger RNA.

These genomic biomarkers were identified by Applicants through gene expression analysis of human SCLC and leukemia/lymphoma cell lines used to test Bcl-2 inhibitors in vitro and in vivo and investigation of their clinical significance. These genomic biomarker combinations are of particular interest for use in companion diagnostic assays to the use of ABT-737 and ABT-263.

Particularly, Applicants identified novel biomarker combinations that discriminate between cell line groups, SCLC (See TABLE 1, TABLE 2 and TABLE 3) and leukemia/lymphoma (See TABLE 4, TABLE 5 and TABLE 6) showing sensitivity and resistance to ABT-737.

### SCLC ABT-737 BIOMARKER SIGNATURE SET

TABLE 1

| Affymetrix ID | Gene Name | Genbank | Description |
|---|---|---|---|
| 201590_x_at | ANXA2 | NM 004039 | annexin A2 |
| 210427_x_at | ANXA2 | BC001388 | annexin A2 |
| 213503_x_at | ANXA2 | BE908217 | annexin A2 |
| 204693_at | CDC42EP1 | NM 007061 | CDC42 effector protein (Rho GTPase binding) 1 |
| 201605_x_at | CNN2 | NM 004368 | calponin 2 |
| 202894_at | EPHB4 | NM 004444 | EPH receptor B4 |
| 203989_x_at | F2R | NM 001992 | coagulation factor II (thrombin) receptor |
| 210220_at | FZD2 | L37882 | frizzled homolog 2 (Drosophila) |
| 202382_s_at | GNPDA1 | NM 005471 | glucosamine-6-phosphate deaminase 1 |
| 215489_x_at | HOMER3 | AI871287 | homer homolog 3 (Drosophila) |
| 210605_s_at | MFGE8 | BC003610 | milk fat globule-EGF factor 8 protein |
| 204880_at | MGMT | NM 002412 | O-6-methylguanine-DNA methyltransferase |
| 203434_s_at | MME | NM 007287 | membrane metallo-endopeptidase (neutral endopeptidase, |
| | | | enkephalinase, CALLA, CD10) |
| 202443_x_at | NOTCH2 | AA291203 | Notch homolog 2 (Drosophila) |
| 210756_s_at | NOTCH2 | AF308601 | Notch homolog 2 (Drosophila) /// Notch homolog 2 |
| | | | (Drosophila) |
| 212377_s_at | NOTCH2 | AU158495 | Notch homolog 2 (Drosophila) |
| 214722_at | NOTCH2NL | AW516297 | Notch homolog 2 (Drosophila) N-terminal like |
| 205503_at | PTPN14 | NM 005401 | protein tyrosine phosphatase, non-receptor type 14 |
| 212262_at | QKI | AA149639 | quaking homolog, KH domain RNA binding (mouse) |
| 205228_at | RBMS2 | NM 002898 | RNA binding motif, single stranded interacting protein 2 |
| 221016_s_at | TCF7L1 | NM 031283 | transcription factor 7-like 1 (T-cell specific, HMG-box) |
| 212761_at | TCF7L2 | AI949687 | transcription factor 7-like 2 (T-cell specific, HMG-box) |
| 212762_s_at | TCF7L2 | AI375916 | transcription factor 7-like 2 (T-cell specific, HMG-box) |
| 216035_x_at | TCF7L2 | AV721430 | transcription factor 7-like 2 (T-cell specific, HMG-box) |
| 216037_x_at | TCF7L2 | AA664011 | transcription factor 7-like 2 (T-cell specific, HMG-box) |
| 216511_s_at | TCF7L2 | AJ270770 | transcription factor 7-like 2 (T-cell specific, HMG-box) |
| 200931_s_at | VCL | NM 014000 | Vinculin |
| 201426_s_at | VIM | AI922599 | Vimentin |
| 202133_at | WWTR1 | BF674349 | WW domain containing transcription regulator 1 |
| 211962_s_at | ZFP36L1 | BG250310 | zinc finger protein 36, C3H type-like 1 |

TABLE 2

| Affymetrix ID | Gene Name | Genbank | Description |
|---|---|---|---|
| 202382_s_at | GNPDA1 | NM 005471 | glucosamine-6-phosphate deaminase 1 |
| 202443_x_at | NOTCH2 | AA291203 | Notch homolog 2 (Drosophila) |
| 202894_at | EPHB4 | NM 004444 | EPH receptor B4 |
| 210220_at | FZD2 | L37882 | frizzled homolog 2 (Drosophila) |
| 210756_s_at | NOTCH2 | AF308601 | Notch homolog 2 (Drosophila) /// Notch homolog 2 |
| | | | (Drosophila) |
| 212377_s_at | NOTCH2 | AU158495 | Notch homolog 2 (Drosophila) |
| 214722_at | NOTCH2NL | AW516297 | Notch homolog 2 (Drosophila) N-terminal like |
| 221016_s_at | TCF7L1 | NM 031283 | transcription factor 7-like 1 (T-cell specific, HMG-box) |

TABLE 3

| | Gene | | |
|---|---|---|---|
| Affymetrix ID | Name | Genbank | Description |
| 200670_at | XBP1 | NM_005080 | X-box binding protein 1 |
| 201012_at | ANXA1 | NM_000700 | annexin A1 |
| 201215_at | PLS3 | NM_005032 | plastin 3 (T isoform) |
| 201387_s_at | UCHL1 | NM_004181 | ubiquitin carboxyl-terminal |
| | | | esterase L1 (ubiquitin |
| | | | thiolesterase) |
| 206502_s_at | INSM1 | NM_002196 | insulinoma-associated 1 |
| 208782_at | FSTL1 | BC000055 | follistatin-like 1 |
| 210715_s_at | SPINT2 | AF027205 | serine peptidase inhibitor, |
| | | | Kunitz type, 2 |
| 211984_at | CALM1 | AI653730 | calmodulin 1 (phosphorylase |
| | | | kinase, delta) |
| 213503_x_at | ANXA2 | BE908217 | annexin A2 |
| 216379_x_at | CD24 | AK000168 | CD24 molecule |

### LEUKEMIA/LYMPHOMA ABT-737 BIOMARKER SIGNATURE SET

TABLE 4

| | Gene | | |
|---|---|---|---|
| Affymetrix ID | Name | Genbank | Description |
| 201118_at | PGD | NM 002631 | phosphogluconate dehydrogenase /// |
| | | | phosphogluconate dehydrogenase |
| 202779_s_at | UBE2S | NM 014501 | Ubiquitin-conjugating enzyme E2S |
| 202950_at | CRYZ | NM 001889 | crystallin, zeta (quinone reductase) |
| 203040_s_at | HMBS | NM 000190 | hydroxymethylbilane synthase |
| 203810_at | DNAJB4 | BG252490 | DnaJ (Hsp40) homolog, subfamily B, member 4 |
| 203911_at | RAP1GA1 | NM 002885 | RAP1, GTPase activating protein 1 |
| 203925_at | GCLM | NM 002061 | glutamate-cysteine ligase, modifier subunit |
| 203946_s_at | ARG2 | NM 001172 | Arginase, type II |
| 204624_at | ATP7B | NM 000053 | ATPase, Cu++ transporting, beta polypeptide |
| | | | (Wilson disease) |
| 205164_at | GCAT | NM 014291 | glycine C-acetyltransferase (2-amino- |
| | | | 3-ketobutyrate coenzyme A ligase) |
| 205262_at | KCNH2 | NM 000238 | potassium voltage-gated channel, |
| | | | subfamily H (eag-related), member 2 |
| 205486_at | TESK2 | NM 007170 | testis-specific kinase 2 |
| 206283_s_at | TAL1 | NM 003189 | T-cell acute lymphocytic leukemia 1 |
| 206729_at | TNFRSF8 | NM 001243 | tumor necrosis factor receptor superfamily, member 8 |
| 207522_s_at | ATP2A3 | NM 005173 | ATPase, Ca++ transporting, ubiquitous |
| 208398_s_at | TBPL1 | NM 004865 | TBP-like 1 |
| 209368_at | EPHX2 | AF233336 | epoxide hydrolase 2, cytoplasmic |
| 210036_s_at | KCNH2 | AB044806 | potassium voltage-gated channel, |
| | | | subfamily H (eag-related), member 2 |
| 211673_s_at | MOCS1 | AF034374 | molybdenum cofactor synthesis 1 /// |
| | | | molybdenum cofactor synthesis 1 |
| 212475_at | KIAA0241 | AI797458 | KIAA0241 protein |
| 213036_x_at | ATP2A3 | Y15724 | ATPase, Ca++ transporting, ubiquitous |
| 214696_at | MGC14376 | AF070569 | hypothetical protein MGC14376 |
| 215150_at | YOD1 | AF090896 | YOD1 OTU deubiquinating enzyme |
| | | | 1 homolog (yeast) |
| 215535_s_at | AGPAT1 | AF007145 | 1-acylglycerol-3-phosphate O-acyltransferase |
| | | | 1 (lysophosphatidic acid acyltransferase, alpha) |
| 216317_x_at | RHCE | X63095 | Rhesus blood group, CcEe antigens |
| 218157_x_at | CDC42SE1 | NM 020239 | CDC42 small effector 1 |
| 218617_at | TRIT1 | NM 017646 | tRNA isopentenyltransferase 1 |
| 218647_s_at | YRDC | BE464161 | Ischemia/reperfusion inducible protein |
| 218739_at | ABHD5 | NM 016006 | abhydrolase domain containing 5 |
| 219103_at | DDEFL1 | NM 017707 | development and differentiation enhancing factor-like 1 |
| 219578_s_at | CPEB1 | AF329403 | cytoplasmic polyadenylation element binding protein 1 |
| 219611_s_at | CCDC21 | NM 022778 | coiled-coil domain containing 21 |
| 219786_at | MTL5 | NM 004923 | metallothionein-like 5, testis-specific (tesmin) |
| 220150_s_at | C6orf60 | NM 024581 | chromosome 6 open reading frame 60 |
| 220399_at | FLJ22639 | NM 024796 | hypothetical protein FLJ22639 |
| 220807_at | HBQ1 | NM 005331 | hemoglobin, theta 1 /// hemoglobin, theta 1 |
| 221693_s_at | MRPS18A | AB049952 | mitochondrial ribosomal protein S18A /// |
| | | | mitochondrial ribosomal protein S18A |
| 32836_at | AGPAT1 | U56417 | 1-acylglycerol-3-phosphate O-acyltransferase |
| | | | 1 (lysophosphatidic acid acyltransferase, alpha) |
| 217862_at | PIAS1 | N24868 | protein inhibitor of activated STAT, 1 |
| 216221_s_at | PUM2 | D87078 | pumilio homolog 2 (Drosophila) |

TABLE 5

| Affymetrix ID | Gene Name | Genbank | Description |
|---|---|---|---|
| 202950_at | CRYZ | NM | crystallin, zeta (quinone reductase) |
| | | 001889 | |
| 203040_s_at | HMBS | NM | hydroxymethylbilane synthase |
| | | 000190 | |
| 205262_at | KCNH2 | NM | potassium voltage-gated channel, subfamily |
| | | 000238 | H (eag-related), member 2 |
| 215150_at | YOD1 | AF090896 | YOD1 OTU deubiquinating enzyme |
| | | | 1 homolog (yeast) |
| 216221_s_at | PUM2 | D87078 | pumilio homolog 2 (Drosophila) |
| 218617_at | TRIT1 | NM | tRNA isopentenyltransferase 1 |
| | | 017646 | |
| 218647_s_at | YRDC | BE464161 | ischemia/reperfusion |
| | | | inducible protein |
| 220399_at | FLJ22639 | NM | hypothetical protein FLJ22639 |
| | | 024796 | |

TABLE 6

| Affymetrix ID | Gene Name | Genbank | Description |
|---|---|---|---|
| 201227_s_at | NDUFB8 | NM_005004 | NADH dehydrogenase (ubiquinone) 1 beta |
| | | | subcomplex, 8, 19 kDa |
| 206283_s_at | TAL1 | NM_003189 | T-cell acute lymphocytic leukemia 1 |
| 207168_s_at | H2AFY | NM_004893 | H2A histone family, member Y |
| 208235_x_at | GAGE7 | NM_021123 | G antigen 7 |
| 209377_s_at | HMGN3 | AF274949 | high mobility group nucleosomal binding domain 3 |
| 211911_x_at | HLA-B | L07950 | major histocompatibility complex, class I, B |
| 213515_x_at | HBG2 | AI133353 | Hemoglobin, gamma G |
| 214039_s_at | LAPTM4B | T15777 | lysosomal associated protein transmembrane 4 beta |
| 216442_x_at | FN1 | AK026737 | fibronectin 1 |
| 216526_x_at | HLA-C | AK024836 | major histocompatibility complex, class I, C |

Applicants further identified biomarker combinations that show sensitivity and resistance to ABT-263 and further discriminating between cell lines, SCLC (See TABLE 7, TABLE 8, TABLE 9, TABLE 10, TABLE 11, TABLE 12 and TABLE 13) and leukemia/lymphoma (See TABLE 14, TABLE 15, TABLE 16, TABLE 17, TABLE 18, TABLE 19 and TABLE 20).

### SCLC ABT-263 BIOMARKER SIGNATURE SET

TABLE 7

| | Gene | | |
|---|---|---|---|
| Affymetrix ID | Name | Genbank | Description |
| 210605_s_at | MFGE8 | BC003610 | milk fat globule-EGF factor |
| | | | 8 protein |
| 202443_x_at | NOTCH2 | NM 024408 | Notch homolog 2 |
| | | | (Drosophila) |
| 203435_s_at | MME | NM 007287 | membrane metallo- |
| | | | endopeptidase (neutral |
| | | | endopeptidase, enkephalinase, |
| | | | CALLA, CD10) |
| 210220_at | FZD2 | L37882 | frizzled homolog 2 |
| | | | (Drosophila) |

TABLE 8

| | Gene | | |
|---|---|---|---|
| Affymetrix ID | Name | Genbank | Description |
| 202499_s_at | SLC2A3 | NM | solute carrier family 2 |
| | | 006931 | (facilitated glucose |
| | | | transporter), member 3 |
| 221016_s_at | TCF7L1 | NM | Transcription factor 7-like 1 |
| | | 031283 | (T-cell specific, HMG-box) |
| 217730_at | TMBIM1 | NM | transmembrane BAX inhibitor |
| | | 022152 | motif containing 1 |
| 218865_at | MOSC1 | NM 022746 | MOCO sulphurase C-terminal |
| | | | domain containing 1 |

TABLE 9

| | Gene | | |
|---|---|---|---|
| Affymetrix ID | Name | Genbank | Description |
| 202443_x_at | NOTCH2 | AA291203 | Notch homolog 2 |
| | | | (Drosophila) |
| 202499_s_at | SLC2A3 | NM | solute carrier family 2 |
| | | 006931 | (facilitated glucose |
| | | | transporter), member 3 |
| 203435_s_at | MME | NM | membrane metallo- |
| | | 007287 | endopeptidase (neutral |
| | | | endopeptidase, enkephalinase, |
| | | | CALLA, CD10) |
| 210220_at | FZD2 | L37882 | frizzled homolog 2 |
| | | | (Drosophila) |
| 210605_s_at | MFGE8 | BC003610 | milk fat globule-EGF factor |
| | | | 8 protein |
| 217730_at | TMBIM1 | NM | transmembrane BAX inhibitor |
| | | 022152 | motif containing 1 |
| 218865_at | MOSC1 | NM 022746 | MOCO sulphurase C-terminal |
| | | | domain containing 1 |

TABLE 10

| | Gene | | |
|---|---|---|---|
| Affymetrix ID | Name | Genbank | Description |
| 200797_s_at | MCL1 | AI275690 | myeloid cell leukemia |
| | | | sequence 1 (BCL2-related) |
| 203684_s_at | BCL2 | M13994 | B-cell CLL/lymphoma 2 |
| 203685_at | BCL2 | NM_000633 | B-cell CLL/lymphoma 2 |
| 204285_s_at | PMAIP1 | AI857639 | Phorbol-12-myristate-13- |
| | | | acetate-induced protein 1 |
| 204286_s_at | PMAIP1 | NM_021127 | phorbol-12-myristate- |
| | | | 13-acetate-induced protein 1 |
| 211725_s_at | BID | BC005884 | BH3 interacting domain |
| | | | death agonist |

TABLE 11

| | Gene | | |
|---|---|---|---|
| Affymetrix ID | Name | Genbank | Description |
| 200797_s_at | MCL1 | AI275690 | Myeloid cell leukemia |
| | | | sequence 1 (BCL2-related) |
| 201231_s_at | ENO1 | NM_001428 | Enolase 1, (alpha) |
| 203685_at | BCL2 | NM_000633 | B-cell CLL/lymphoma 2 |
| 204285_s_at | PMAIP1 | AI857639 | Phorbol-12-myristate-13- |
| | | | acetate-induced protein 1 |
| 204798_at | MYB | NM_005375 | v-myb myeloblastosis viral |
| | | | oncogene homolog (avian) |
| 208727_s_at | CDC42 | BC002711 | cell division cycle 42 (GTP |
| | | | binding protein, 25 kDa) |
| 209397_at | ME2 | BC000147 | malic enzyme 2, NAD(+)- |
| | | | dependent, mitochondrial |
| 211275_s_at | GYG1 | AF087942 | glycogenin 1 |
| 211474_s_at | SERPINB6 | BC004948 | serpin peptidase inhibitor, |
| | | | clade B (ovalbumin), |
| | | | member 6 |
| 216623_x_at | TOX3 | AK025084 | TOX high mobility group |
| | | | box family member 3 |

TABLE 12

| | Gene | | |
|---|---|---|---|
| Affymetrix ID | Name | Genbank | Description |
| 202443_x_at | NOTCH2 | AA291203 | Notch homolog 2 |
| | | | (Drosophila) |
| 203684_s_at | BCL2 | M13994 | B-cell CLL/lymphoma 2 |
| 203685_at | BCL2 | NM_000633 | B-cell CLL/lymphoma 2 |
| 204285_s_at | PMAIP1 | AI857639 | Phorbol-12-myristate-13- |
| | | | acetate-induced Protein 1 |
| 204286_s_at | PMAIP1 | NM_021127 | phorbol-12-myristate-13- |
| | | | acetate-induced protein 1 |
| 210220_at | FZD2 | L37882 | Frizzled homolog 2 |
| | | | (Drosophila) |
| 218865_at | MOSC1 | NM_022746 | MOCO sulphurase C- |
| | | | terminal domain containing 1 |

TABLE 13

| | Gene | | |
|---|---|---|---|
| Affymetrix ID | Name | Genbank | Description |
| 200872_at | S100A10 | NM_002966 | S100 calcium binding protein |
| | | | A10 |
| 201105_at | LGALS1 | NM_002305 | Lectin, galactoside-binding, |
| | | | soluble, 1 (galectin 1) |
| 201231_s_at | ENO1 | NM_001428 | enolase 1, (alpha) |
| 201477_s_at | RRM1 | NM_001033 | ribonucleotide reductase |
| | | | M1 polypeptide |
| 202088_at | SLC39A6 | AI635449 | solute carrier family 39 (zinc |
| | | | transporter), member 6 |
| 209366_x_at | CYB5A | M22865 | cytochrome b5 type A |
| | | | (microsomal) |
| 211528_x_at | HLA-G | M90685 | major histocompatibility |
| | | | complex, class I, G |
| 212063_at | CD44 | BE903880 | CD44 molecule (Indian blood |
| | | | group) |
| 216623_x_at | TOX3 | AK025084 | TOX high mobility group box |
| | | | family member 3 |
| 217294_s_at | ENO1 | U88968 | enolase 1, (alpha) |

### LEUKEMIA/LYMPHOMA ABT-263 BIOMARKER SIGNATURE SET

TABLE 14

| Affymetrix ID | Gene Name | Genbank | Description |
|---|---|---|---|
| 201828_x_at | CXX1 | NM 003928 | CAAX box 1 |
| 205691_at | SYNGR3 | NM 004209 | synaptogyrin 3 |
| 208796_s_at | CCNG1 | BC000196 | cyclin G1 |
| 214696_at | MGC14376 | AF070569 | hypothetical protein |
| | | | MGC14376 |
| 220051_at | PRSS21 | NM 006799 | protease, serine, 21 |
| | | | (testisin) |

TABLE 15

| Affymetrix ID | Gene Name | Genbank | Description |
|---|---|---|---|
| 210775_x_at | CASP9 | AB015653 | caspase 9, apoptosis-related |
| | | | cysteine peptidase |
| 211560_s_at | ALAS2 | AF130113 | aminolevulinate, delta-, |
| | | | synthase 2 (sideroblastic/ |
| | | | hypochromic anemia) |
| 217650_x_at | ST3GAL2 | AI088162 | ST3 beta-galactoside alpha- |
| | | | 2,3-sialyltransferase 2 |
| 217955_at | BCL2L13 | NM 015367 | BCL2-like 13 (apoptosis |
| | | | facilitator) |
| 204517_at | PPIC | BE962749 | peptidylprolyl isomerase C |
| | | | (cyclophilin C) |
| 201559_s_at | CLIC4 | AF109196 | chloride intracellular |
| | | | channel 4 |
| 208398_s_at | TBPL1 | NM 004865 | TBP-like 1 |
| 209116_x_at | HBB | M25079 | hemoglobin, beta /// |
| | | | hemoglobin, beta |
| 207180_s_at | HTATIP2 | NM 006410 | HIV-1 Tat interactive |
| | | | protein 2, 30 kDa |

TABLE 16

| | Gene | | |
|---|---|---|---|
| Affymetrix ID | Name | Genbank | Description |
| 201828_x_at | CXX1 | NM 003928 | CAAX box 1 |
| 205691_at | SYNGR3 | NM 004209 | synaptogyrin 3 |
| 207180_s_at | HTATIP2 | NM 006410 | HIV-1 Tat interactive |
| | | | protein 2, 30 kDa |
| 208796_s_at | CCNG1 | BC000196 | cyclin G1 |
| 209116_x_at | HBB | M25079 | hemoglobin, beta /// |
| | | | hemoglobin, beta |
| 211560_s_at | ALAS2 | AF130113 | aminolevulinate, delta-, |
| | | | synthase 2 (sideroblastic/ |
| | | | hypochromic anemia) |
| 214696_at | MGC14376 | AF070569 | hypothetical protein |
| | | | MGC14376 |
| 217650_x_at | ST3GAL2 | AI088162 | ST3 beta-galactoside alpha- |
| | | | 2,3-sialyltransferase 2 |

TABLE 17

| Affymetrix ID | Gene Name | Genbank | Description |
|---|---|---|---|
| 200796_s_at | MCL1 | BF594446 | myeloid cell leukemia |
| | | | sequence 1 (BCL2-related) |
| 200797_s_at | MCL1 | AI275690 | myeloid cell leukemia |
| | | | sequence 1 (BCL2-related) |
| 200798_x_at | MCL1 | NM_021960 | myeloid cell leukemia |
| | | | sequence 1 (BCL2-related) |
| 203684_s_at | BCL2 | M13994 | B-cell CLL/lymphoma 2 |
| 204493_at | BID | NM_001196 | BH3 interacting domain |
| | | | death agonist |
| 206665_s_at | BCL2L1 | NM_001191 | BCL2-like 1 |
| 209311_at | BCL2L2 | D87461 | BCL2-like 2 |
| 211692_s_at | BBC3 | AF332558 | BCL2 binding component 3 |

TABLE 18

| | Gene | | |
|---|---|---|---|
| Affymetrix ID | Name | Genbank | Description |
| 200797_s_at | MCL1 | AI275690 | myeloid cell leukemia |
| | | | sequence 1 (BCL2-related) |
| 200798_x_at | MCL1 | NM_021960 | myeloid cell leukemia |
| | | | sequence 1 (BCL2-related) |
| 205691_at | SYNGR3 | NM_004209 | synaptogyrin 3 |
| 207180_s_at | HTATIP2 | NM_006410 | HIV-1 Tat interactive |
| | | | protein 2, 30 kDa |
| 208796_s_at | CCNG1 | BC000196 | cyclin G1 |
| 211560_s_at | PRO2399 | AF130113 | Homo sapiens clone FLB8929 |
| | | | PRO2399 mRNA, complete |
| | | | cds. |
| 214696_at | C17orf91 | AF070569 | chromosome 17 open reading |
| | | | frame 91 |
| 217650_x_at | ST3GAL2 | AI088162 | ST3 beta-galactoside alpha- |
| | | | 2,3-sialyltransferase 2 |

TABLE 19

| Affymetrix ID | Gene Name | Genbank | Description |
|---|---|---|---|
| 200798_x_at | MCL1 | NM_021960 | myeloid cell leukemia |
| | | | sequence 1 (BCL2-related) |
| 201288_at | ARHGDIB | NM_001175 | Rho GDP dissociation |
| | | | inhibitor (GDI) beta |
| 202207_at | ARL4C | BG435404 | ADP-ribosylation factor- |
| | | | like 4C |
| 203408_s_at | SATB1 | NM_002971 | SATB homeobox 1 |
| 203489_at | SIVA1 | NM_006427 | SIVA1, apoptosis-inducing |
| | | | factor |
| 203685_at | BCL2 | NM_000633 | B-cell CLL/lymphoma 2 |
| 205681_at | BCL2A1 | NM_004049 | BCL2-related protein A1 |
| 205919_at | HBE1 | NM_005330 | hemoglobin, epsilon 1 |
| 209942_x_at | MAGEA3 | BC000340 | melanoma antigen |
| | | | family A, 3 |
| 211725_s_at | BID | BC005884 | BH3 interacting domain |
| | | | death agonist |

TABLE 20

| Affymetrix ID | Gene Name | Genbank | Description |
|---|---|---|---|
| 201029_s_at | CD99 | NM_002414 | CD99 molecule |
| 201288_at | ARHGDIB | NM_001175 | Rho GDP dissociation |
| | | | inhibitor (GDI) beta |
| 201310_s_at | C5orf13 | NM_004772 | chromosome 5 open |
| | | | reading frame 13 |
| 201347_x_at | GRHPR | NM_012203 | glyoxylate reductase/ |
| | | | hydroxypyruvate reductase |
| 206660_at | IGLL1 | NM_020070 | immunoglobulin lambda- |
| | | | like polypeptide 1 |
| 208892_s_at | DUSP6 | BC003143 | dual specificity |
| | | | phosphatase 6 |
| 209806_at | HIST1H2BK | BC000893 | histone cluster 1, H2bk |
| 209942_x_at | MAGEA3 | BC000340 | melanoma antigen |
| | | | family A, 3 |
| 211921_x_at | PTMA | AF348514 | prothymosin, alpha |
| | | | (gene sequence 28) |
| 213515_x_at | HBG2 | AI133353 | Hemoglobin, gamma G |

III. Assays

The inventive assays include assays both to select patients eligible to receive Bcl-2 family inhibitor therapy and assays to monitor patient response. Assays for response prediction are run before therapy selection and patients with elevated levels are eligible to receive Bcl-2 family inhibitor therapy. For monitoring patient response, the assay is run at the initiation of therapy to establish baseline (or predetermined) levels of the biomarker in the tissue sample. The same tissue is then sampled and assayed and the levels of the biomarker compared to the baseline or predetermined levels. The comparison (or informational analysis) of the level of the assayed biomarker with the baseline or predetermined level can be done by an automated system, such as a software program or intelligence system that is part of, or compatible with, the equipment (e.g., computer platform) on which the assay is carried out. Alternatively, this comparison or informational analysis can be done by a physician. In those instances where the levels remain the same or decrease, the therapy is likely being effective and can be continued. Where significant increase over baseline level (or predetermined level) occurs, the patient may not be responding.

The assays of the present invention can be performed by protein assay methods and by nucleic acid assay methods. Any type of either protein or nucleic acid assays can be used. Protein assay methods useful in the invention are well known in the art and comprise (i) immunoassay methods involving binding of a labeled antibody or protein to the expressed protein or fragment of genes in the biomarker set, (ii) mass spectrometry methods to determine expressed protein or fragments of these biomarkers, and (iii) proteomic based or “protein chip” assays. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

IHC methods are particularly preferred assays. IHC is a method of detecting the presence of specific proteins in cells or tissues and consists of the following steps: 1) a slide is prepared with the tissue to be interrogated; 2) a primary antibody is applied to the slide and binds to specific antigen; 2) the resulting antibody-antigen complex is bound by a secondary, enzyme-conjugated, antibody; 3) in the presence of substrate and chromogen, the enzyme forms a colored deposit (a “stain”) at the sites of antibody-antigen binding; and 4) the slide is examined under a microscope to identify the presence of and extent of the stain.

Nucleic acid assay methods useful in the invention are also well known in the art and comprise (i) in situ hybridization assays to intact tissue or cellular samples to detect mRNA levels, (ii) microarray hybridization assays to detect mRNA levels, (iii) RT-PCR assays or other amplification assays to detect mRNA levels. Assays using synthetic analogs of nucleic acids, such as peptide nucleic acids, in any of these formats can also be used.

The assay of the present invention also provide for detection of the genomic biomarkers by hybridization assays using detectably labeled nucleic acid-based probes, such as deoxyribonucleic acid (DNA) probes or protein nucleic acid (PNA) probes, or unlabeled primers which are designed/selected to hybridize to the specific designed gene target. The unlabeled primers are used in amplification assays, such as by polymerase chain reaction (PCR), in which after primer binding, a polymerase amplifies the target nucleic acid sequence for subsequent detection. The detection probes used in PCR or other amplification assays are preferably fluorescent, and still more preferably, detection probes useful in “real-time PCR”. Fluorescent labels are also preferred for use in situ hybridization but other detectable labels commonly used in hybridization techniques, e.g., enzymatic, chromogenic and isotopic labels, can also be used. Useful probe labeling techniques are described in Molecular Cytogenetics: Protocols and Applications, Y.-S. Fan, Ed., Chap. 2, “Labeling Fluorescence In Situ Hybridization Probes for Genomic Targets”, L. Morrison et.al., p. 21-40, Humana Press, © 2002, incorporated herein by reference.

A further embodiment the gene expression levels of the biomarker combinations set forth in TABLES 1, 2, 3, 4, 5, 6, 7, 8, 9, 10, 11, 12, 13, 14, 15, 16, 17, 18, 19 or 20 can be evaluated using nucleic acid based arrays such as for example cDNA or oligonucleotide arrays, or protein arrays.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectric, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides prepared from one sample, such as peripheral blood, tumor or suspected tumor tissues, or cell isolated such as circulating epithelial cells separated or identified in a blood sample, at a specific time during the course of an anti-cancer treatment, are hybridized to a nucleic acid array. Signals detected after the formation of hybridization complexes indicate that polynucleotide levels in the sample. In one embodiment, the fluorophores Cy3 and Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetric or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained n each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

IV. Sample Processing and Assay Performance

The tissue sample to be assayed by the inventive methods can comprise any type, including a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, and this extract can then be assayed. A microdissection of the tissue sample to obtain a cellular sample enriched with suspected tumor cells can also be used. The preferred tissue samples for use herein are peripheral blood, tumor tissue or suspected tumor tissue, including fine needle aspirates, fresh frozen tissue and paraffin embedded tissue, and bone marrow.

The tissue sample can be processed by any desirable method for performing in situ hybridization or other nucleic acid assays. For the preferred in situ hybridization assays, a paraffin embedded tumor tissue sample or bone marrow sample is fixed on a glass microscope slide and deparaffinized with a solvent, typically xylene. Useful protocols for tissue deparaffinization and in situ hybridization are available from Abbott Molecular Inc. (Des Plaines, Ill.). Any suitable instrumentation or automation can be used in the performance of the inventive assays. PCR based assays can be performed on the m2000 instrument system (Abbott Molecular, Des Plaines, Ill.). Automated imaging can be employed for the preferred fluorescence in situ hybridization assays.

In one embodiment, the sample comprises a peripheral blood sample from a patient which is processed to produce an extract of circulating tumor cells having increased expression of the biomarker genes. The circulating tumor cells can be separated by immunomagnetic separation technology such as that available from Immunicon (Huntingdon Valley, Pa.). The number of circulating tumor cells showing altered expression of biomarker genes is then compared to the baseline level of circulating tumor cells having altered expression of biomarker genes determined preferably at the start of therapy.

Test samples can comprise any number of cells that is sufficient for a clinical diagnosis, and typically contain at least about 100 cells.

V. Assay Kits

In another aspect, the invention comprises immunoassay kits for the detection of which kits comprise a labeled antibody or labeled protein specific for binding to genes in the biomarkers set. These kits may also include an antibody capture reagent or antibody indicator reagent useful to carry out a sandwich immunoassay. Preferred kits of the invention comprise containers containing, respectively, at least one antibody capable of binding specifically to at least one of the biomarkers in the set, and a control gene. Any suitable control composition for the particular biomarker assay can be included in the kits of the invention. The control compositions generally comprise the biomarker to be assayed for, along with any desirable additives. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding the genes set forth in the biomarker combinations of the present invention. Such kits generally comprise at least one oligonucleotide probe or primer, and preferably oligonucleotide sets corresponding to the biomarker combination groups set out in TABLES 1, 2, 3, 4, 5, 6, 7, 8, 9, 10, 11, 12, 13, 14, 15, 16, 17, 18, 19 or 20 as described above that hybridizes to a polynucleotide encoding a protein. Such oligonucleotides may be used, for example, within a PCR or hybridization assay. Additional components that may be present within such kits include a second oligonucleotide, or a set of oligonucleotides corresponding to the biomarker combinations set out in TABLES 1, 2, 3, 4, 5, 6, 7, 8, 9, 10, 11, 12, 13, 14, 15, 16, 17, 18, 19 or 20, and/or a diagnostic reagent or container to facilitate the detection of a polynucleotide encoding a tumor protein.

VI. Databases

In yet a further aspect the invention includes relational databases containing sequence information, for instance for one or more of the genes of TABLES 1, 2, 3, 4, 5, 6, 7, 8, 9, 10, 11, 12, 13, 14, 15, 16, 17, 18, 19 or 20, as well as gene expression information in various lung cancer and leukemia/lymphoma tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. The databases of the invention may be stored on any available computer-readable medium. Methods for the configuration and construction of such databases are widely available, for instance, see Akerblom et al., (U.S. Pat. No. 5,953,727), which is specifically incorporated herein by reference in its entirety.

The databases of the invention may be linked to an outside or external database. In a preferred embodiment, as described in Tables 1, 2, 3, 4, 5, 6, 7, 8, 9, 10, 11, 12, 13, 14, 15, 16, 17, 18, 19 or 20 the external database is GenBank and the associated databases maintained by the National Center for Biotechnology Information or NCBI. Other external databases that may be used in the invention include those provided by Chemical Abstracts Service or Incyte Genomics.

Any appropriate automated system, such as a software program or intelligence system (e.g., computer platform) may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northern blots (E-Northerns) to allow the user to determine the cell type or tissue in which a given gene is expressed and to allow determination of the abundance or expression level of a given gene in a particular tissue or cell. The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial.

The databases of the invention and optionally, any accompanying automated system, such as a software program or intelligence system (e.g., computer platform), may also be used to present information identifying the expression level in a tissue or cell of a combination of genes set out in TABLES 1, 2, 3, 4, 5, 6, 7, 8, 9, 10, 11, 12, 13, 14, 15, 16, 17, 18, 19 or 20, comprising the step of comparing the expression level of the biomarker combinations set out in TABLES 1, 2, 3, 4, 5, 6, 7, 8, 9, 10, 11, 12, 13, 14, 15, 16, 17, 18, 19 or 20 in the tissue to the level of expression of the gene in the database. Such methods may be used to predict the physiological state of a given tissue by comparing the level of expression of the gene combinations set out in TABLES 1, 2, 3, 4, 5, 6, 7, 8, 9, 10, 11, 12, 13, 14, 15, 16, 17, 18, 19 or 20 from a sample to the expression levels found in tissue from normal tissue, tissue from tumors or both. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The preceding working examples therefore, are illustrative only and should not be construed as limiting in any way the scope of the invention.

VII. Experimental

### Example 1

A genome-wide view of gene expression patterns using microarrays.

### A. Cell Culture.

The following SCLC cell lines were obtained from ATCC (Manassis, Va.): NCI-H889, NCI-H1963, NCI-H1417, NCI-H146, NCI-H187, DMS53, NCI-H510, NCI-H209, NCI-H211, NCI-H345, NCI-H524, NCI-H69, DMS79, SHP77, NCI-H1688, NCI-H446, NCI-H740, NCI-H1048, NCI-H82, NCI-H196, SW1271, H69AR, NCI-H526, NCI-H865, NCI-H748, NCI-H711, and DMS114. All cells were cultured in the ATCC recommended media at 37° C. in a humidified atmosphere containing 5% CO₂. The following leukemia and lymphoma cell lines were obtained from ATCC (Manassis, Va.): MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, Jurkat I 9.2, Jurkat, MEG-01, U-937, K-562, and Raji.

### B. Microarray Analysis of Gene Expression.

Total RNA was isolated by using the Trizol reagent (Invitrogen,) and purified on RNeasy columns (Qiagen, Valencia, Calif.). Labeled cRNA was prepared according to the microarray manufacturer's protocol and hybridized to human U133A 2.0 arrays (Affymetrix, Santa Clara, Calif.). The U133A 2.0 chips contain 14,500 well-characterized genes, as well as several thousand ESTs. The microarray data files were loaded into the Rosetta Resolver™ software for analysis and the intensity values for all probe sets were normalized using the Resolver's Experimental Definition. The intensity values for the probesets corresponding to genes within the amplified regions were normalized across each gene and compared in heatmaps using the Spotfire® software (available from TIBCO®).

### C. Results.

The 27 SCLC cell lines were tested for sensitivity to ABT-737 using the procedure described in Oltersdorf, T., “An inhibitor of Bcl-2 family proteins induces regression of solid tumours”, Nature, 435: 677-681, 2005, with a cell line classified as sensitive if its EC50<5 μM and as resistant if its EC50>5 82 M. The sensitive cell line group consisted of NCI-H889, NCI-H1963, NCI-H1417, NCI-H146, DMS 53, NCI-H187, NCI-H510, NCI-H209, NCI-H345, NCI-H526, NCI-H211, NCI-H865, NCI-H524, NCI-H748, DMS 79, NCI-H69, NCI-H711, SHP 77, NCI-H1688, and and the resistant cell line group was comprised of NCI-H446, NCI-H740, NCI-H1048, NCI-H82, NCI-H196, SW1271, DMS 114, and NCI-H69AR.

The 22 SCLC cell lines were tested for sensitivity to ABT-263 using the procedure described in Oltersdorf, T., “An inhibitor of Bcl-2 family proteins induces regression of solid tumours”, Nature, 435: 677-681, 2005, with a cell line classified as sensitive if its EC50<5 82 M and as resistant if its EC50>5 μM. The sensitive cell line group consisted of NCI-H146, NCI-H889, NCI-H1963, NCI-H187, NCI-H1417, NCI-H211, NCI-H69, NCI-H209, NCI-H510, DMS 53, DMS 79, NCI-H345, NCI-H1048, SHP 77, NCI-H446 and the resistant cell line group was comprised of NCI-H1688, NCI-H740, NCI-H82, NCI-H69AR, SW1271, DMS 114 and NCI-H 196.

The 25 leukemia/lymphoma cell lines were also tested for sensitivity to ABT-737 using the 5 uM cut-off, and sensitive cell lines were MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, and Jurkat I 9.2, and the resistant cell lines Jurkat, MEG-01, U-937, K-562, and Raji.

The 25 leukemia/lymphoma cell lines were also tested for sensitivity to ABT-263 using the 5 uM cut-off, and sensitive cell lines were MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, and Jurkat I 9.2, and the resistant cell lines Jurkat, MEG-01, U-937, K-562, and Raji.

RNA expression patterns from untreated SCLC cell lines and leukemia/lymphoma cell lines were determined using Affymetrix HG-U133A v.2.0 microarrays that contain over 22,000 probe sets. In parallel with separate cultures, we determined the sensitivity of each cell line to the compounds. The expression profiles were divided into sensitive and resistant groups, and a series of statistical filters applied to identify which genes were the best at discriminating between the sensitive and resistant cell lines. The first filter was an Analysis of Variance (ANOVA) using Spotfire® software (available from TIBCO®). Variable genes (high CV) were next filtered. The remaining genes were analyzed with JMP's discriminant analysis function to identify the genes that best discriminated between sensitive and resistant cell lines. The best discriminant sets were tested using SAS's leave-one-out cross validation function to identify the best signature set of biomarkers. For ABT-263, 2 sets for each cell type (SCLC and leukemia/lymphoma cells) were found to perform well.

### Example 2

### A. Cell Culture and Viability Assays.

The SCLC and leukemia/lymphoma cell lines listed below were obtained from the American Type Culture Collection (Manassas, Va.), Deutsche Sammlung von Mikroorganismen und Zellkulturen (DSMZ, Braunsweig, Germany), or were the generous gift from Dr. Louis Staudt (National Cancer Institute) and were cultured as described in Tse, C., Shoemaker, A. R., Adickes, J., et al., “ABT-263: a potent and orally bioavailable Bcl-2 family inhibitor,” Cancer Res 2008; 68: 3421-3428; and Tahir, S. K., Yang, X., Anderson, M. G., et al., “Influence of Bcl-2 family members on the cellular response of small-cell lung cancer cell lines to ABT-737,” Cancer Res 2007; 67: 1176-1183.

SCLC cell lines used were: NCI-H889, NCI-H1963, NCI-H1417, NCI-H146, NCI-H187, DMS53, NCI-H510, NCI-H209, NCI-H211, NCI-H345, NCI-H524, NCI-H69, DMS79, SHP77, NCI-H1688, NCI-H446, NCI-H740, NCI-H1048, NCI-H82, NCI-H196, SW1271, H69AR, NCI-H526, NCI-H865, NCI-H748, NCI-H711, DMS114, NCI-H847, NCI-H2107, NCI-H1836, NCI-H1105, NCI-H1672, NCI-H1436, NCI-H1618, NCI-H128, NCI-H1930, NCI-H1694, DMS-153, NCI-H2081, and NCI-H378.

Leukemia and lymphoma cell lines used were: MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, Jurkat I 9.2, Jurkat, MEG-01, U-937, K-562, Raji, HBL-2, Granta, Mino, REC-1, SP53, JeKo-1, JVM-2, and JVM-13.

Cells were treated at 10,000 or 50,000 cells per well for adherent or suspension cells, respectively, in 96-well microtiter plates in the presence of 10% human serum for 48 hours with or without ABT-263, in a humidified chamber with 5% CO₂. ABT-263 was synthesized as previously described in Tse, C., Shoemaker, A. R., Adickes, J., et al., “ABT-263: a potent and orally bioavailable Bcl-2 family inhibitor,” Cancer Res 2008; 68: 3421-3428; and Oltersdorf, T., Elmore, S. W., Shoemaker, A. R., et al., “An inhibitor of Bcl-2 family proteins induces regression of solid tumours,” Nature 2005; 435: 677-681, and cell cytotoxicity EC₅₀ values were assessed using CellTiter Glo (Promega, Madison Wis.).

### B. RNA Isolation and Microarrays.

RNA from 8 SCLC tumor samples, (Catalogue number R8235152-PT (lot numbers A805144, A701047, A701062, A701046, A805145, A701061, A610276, and A609162) and 6 matched normal adjacent tissue (lot numbers A701047, A701062, A701046, A805145, A701061, and A610276) were all purchased from Biochain (Hayward, Calif.). Naive cell line samples were lysed and total RNA was isolated using TRIzol reagent (Invitrogen, Carlsbad, Calif.) and purified on RNeasy columns (Qiagen, Valencia, Calif.). To increase statistical power, replicates (between 2 and 4) of each cell line were grown in parallel and analyzed separately. Labeled cRNA was prepared according to the microarray manufacturer's protocol and hybridized to human U133A 2.0 arrays (Affymetrix, Santa Clara, Calif.). The microarray data files were loaded into Rosetta Resolver (Rosetta Biosoftware, Seattle, Wash.) software for analysis, and the intensity values for all probe sets were normalized using Resolver's Experimental Definition tool.

### C. Statistical Analysis.

o divide the training set cell lines into sensitive and resistant categories, a bivariate fit curve was generated for the ABT-263 EC₅₀ versus rank and fitted with a smoothing spline (lambda=1), and the maximum increase in the slope was approximately 5 μM for both sets. This segregated the SCLC lines into 26 sensitive and 10 resistant lines, and the leukemia/lymphoma cell lines into 25 sensitive and 6 resistant cell lines. To compare the expression of the small subset of genes related to the target (Bcl-2 family members), a 0.05 p-value filter was used, with the expression data from all cell lines. To identify global expression markers, expression ratios were made comparing the sensitive to the resistant lines within Resolver's Experimental Definition tool, and then sorted based on p-value. The top 100 probe sets that varied between the sensitive and resistant cell lines by ANOVA were further filtered by Discriminant Analysis in JMP (version 6.0, SAS, Cary, N.C.) to identify the best group of probe sets for predicting sensitivity and resistance to ABT-263. These groups were further tested by a leave-one-out cross validation in SAS and an error rate was calculated for the cell lines that were left out each time. As a validation step, an additional test set of 14 SCLC cell lines (10 sensitive and 4 resistant) was profiled as the other lines were and tested in Discriminant Analysis as unknowns. A similar process was done with a panel of 9 Mantle Cell Lymphomas (8 sensitive, 1 resistant).

To use the entire data set to identify additional gene signatures, several derivation procedures and model fitting algorithms (Random Forests, Bayesian Trees, Neural Nets, and Support Vector Machines) were tested. Overall, based on accuracy of prediction, the performance of signatures were found to be optimal from the Diagonal Linear Discriminant Analysis (DLDA) with simulated annealing algorithm. Using this approach, genes that were significant on their own based on permutation-based Wilcoxon-test in the significance analysis of microarrays (SAM) method were first identified prior to deriving the signatures. This entire procedure of filtering out the important genes, deriving the optimal signature, and model fitting was evaluated using 10 replications of 5-fold stratified cross-validation. In the 5-fold stratified cross-validation procedure, the cell-lines were randomly divided into five equal parts (folds) and stratified to ensure approximately similar prevalence of resistant and non-resistance cell lines within each fold. Each fold was left out one at a time while the gene filtering, signature derivation and model fitting process was carried out in the remaining four parts, and the results were then used to determine whether the cell-lines in the left-out fold were predicted to be resistant or non-resistant. The predictions from each of the left-out folds were then aggregated to determine the overall accuracy of this procedure. This entire evaluation was repeated 10 times, and the mean percentage of cell lines correctly identified from these replications was determined, with the best performing sets reported. This same analysis was then repeated on the leukemia/lymphoma cell lines. All of these analyses were carried out using programs written in R, version 2.7.

### D. Results.

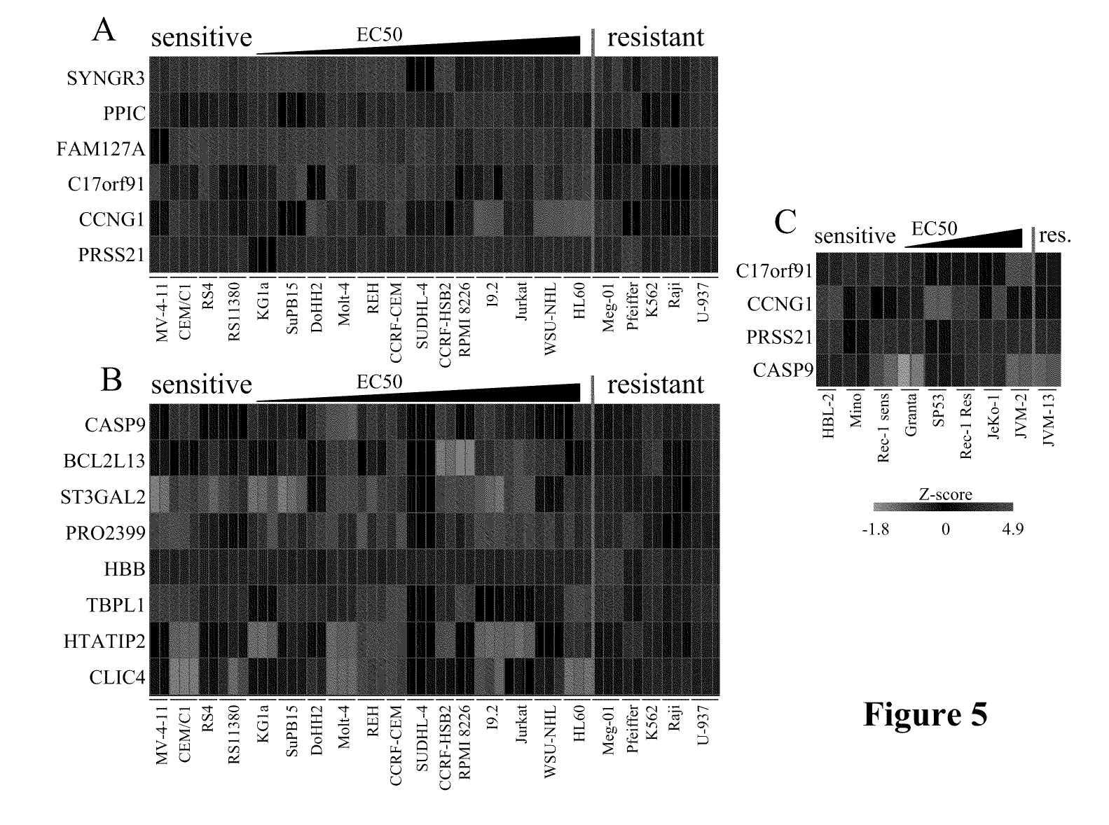

Division of cell lines into sensitive and resistant categories. Markers for sensitivity and resistance to ABT-263 will help identify specific tumors and tumor types where the drug can be more effective, while also identifying additional targets for therapy. To identify gene expression patterns that can predict sensitivity to ABT-263, as well as genes that might contribute to resistance to treatment, RNA expression profiles for a panel of SCLC cell lines were determined, as well as a panel of leukemia and lymphoma cell lines. RNA from untreated cells from these lines was profiled on Affymetrix HGU133A microarrays that contain over 22,000 probe sets. These lines were divided into sensitive and resistant sets by plotting their ABT-263 EC₅₀ values (See, Tse, C., Shoemaker, A. R., Adickes, J., et al., “ABT-263: a potent and orally bioavailable Bcl-2 family inhibitor,” Cancer Res 2008; 68: 3421-3428) versus their rank to identify an EC₅₀ break point. A bivariate fit curve was generated, and the maximum increase in the slope was between the 4.1 μM and 8.1 μM EC₅₀ values for the SCLC panel, and 4.8 μM and 6.0 μM EC₅₀ values for the leukemia/lymphoma panel (FIG. 5). Therefore, SCLC cell lines with EC₅₀ values less than or equal to 4.1 μM and leukemia/lymphoma cell lines with EC₅₀ values less than or equal to 4.8 μM were categorized as sensitive. Using these criteria, 26 out of 36 SCLC cell lines and 25 out of 31 leukemia/lymphoma cell lines were categorized as being sensitive to ABT-263 (Tables A and B).

TABLE A
EC₅₀ values of SCLC cell lines for ABT-263 and
their response designation.

| Cell lines | EC₅₀ (μM) | Response Designation | Set |
|---|---|---|---|
| NCI-H146 | 0.11 | Sensitive | Training |
| NCI-H889 | 0.14 | Sensitive | Training |
| NCI-H1963 | 0.18 | Sensitive | Training |
| NCI-H187 | 0.21 | Sensitive | Training |
| NCI-1417 | 0.36 | Sensitive | Training |
| NCI-211 | 0.41 | Sensitive | Training |
| NCI-H69 | 0.71 | Sensitive | Training |
| NCI-H209 | 1.15 | Sensitive | Training |
| NCI-H510 | 1.22 | Sensitive | Training |
| DMS 53 | 1.6 | Sensitive | Training |
| DMS79 | 1.9 | Sensitive | Training |
| NCI-H345 | 2.16 | Sensitive | Training |
| NCI-1048 | 2.86 | Sensitive | Training |
| SHP-77 | 3.9 | Sensitive | Training |
| NCI-H446 | 4.1 | Sensitive | Training |
| NCI-H1688 | 8.07 | Resistant | Training |
| NCI-H740 | >10 | Resistant | Training |
| NCI-H82 | 22.4 | Resistant | Training |
| H69AR | 22.3 | Resistant | Training |
| SW 1271 | 33.9 | Resistant | Training |
| DMS 114 | 33.9 | Resistant | Training |
| NCI-H196 | 38.6 | Resistant | Training |
| NCI-H847 | 0.06 | Sensitive | Test |
| NCI-H524 | 0.08 | Sensitive | Test |
| NCI-H2107 | 0.3 | Sensitive | Test |
| NCI-H1836 | 0.4 | Sensitive | Test |
| NCI-H1105 | 0.7 | Sensitive | Test |
| NCI-H1672 | 0.7 | Sensitive | Test |
| NCI-H1436 | 1 | Sensitive | Test |
| NCI-H1618 | 1.4 | Sensitive | Test |
| NCI-H128 | 2.5 | Sensitive | Test |
| NCI-H1930 | 3 | Sensitive | Test |
| DMS153 | 11 | Resistant | Test |
| NCI-H2081 | 12 | Resistant | Test |
| NCI-H526 | 12.6 | Resistant | Test |
| NCI-H378 | >50 | Resistant | Test |

TABLE B
EC₅₀ values of leukemia and lymphoma cell lines for
ABT-263 and their response designation.

| | | Response | |
|---|---|---|---|
| Cell lines | EC₅₀ (μM) | Designation | Set |
| MV-4-11 | 0.028 | Sensitive | Training |
| CEM/C1 | 0.063 | Sensitive | Training |
| RS4; 11 | 0.095 | Sensitive | Training |
| RS11380 | 0.145 | Sensitive | Training |
| KG1a | 0.243 | Sensitive | Training |
| SuPB15 | 0.26 | Sensitive | Training |
| DoHH2 | 0.322 | Sensitive | Training |
| Molt-4 | 0.385 | Sensitive | Training |
| REH | 0.434 | Sensitive | Training |
| CCRF-CEM | 0.765 | Sensitive | Training |
| SuDHL-4 | 1.036 | Sensitive | Training |
| CCRF-HSB2 | 1.15 | Sensitive | Training |
| RPMI 8226 | 1.5 | Sensitive | Training |
| I9.2 | 1.8 | Sensitive | Training |
| Jurkat | 3.35 | Sensitive | Training |
| WSU-NHL | 3.55 | Sensitive | Training |
| HL60 | 4.84 | Sensitive | Training |
| Meg-01 | 6.04 | Resistant | Training |
| Pfeiffer | 8.81 | Resistant | Training |
| K562 | 12.8 | Resistant | Training |
| Raji | 23.68 | Resistant | Training |
| U-937 | 33.9 | Resistant | Training |
| HBL-2 | 0.031 | Sensitive | Test |
| Mino | 0.033 | Sensitive | Test |
| Rec-1* | 0.034 | Sensitive | Test |
| Granta | 0.041 | Sensitive | Test |
| SP53 | 0.14 | Sensitive | Test |
| Rec-1* | 0.698 | Sensitive | Test |
| JeKo-1 | 1.743 | Sensitive | Test |
| JVM-2 | 4.11 | Sensitive | Test |
| JVM-13 | 12.97 | Resistant | Test |

*Two different batches of Rec-1 cell line sub-clones had significantly different expression patterns and EC₅₀ values, and were considered separately in the analysis.

### E. Correlation of Bcl-2 Family Member Expression Levels and Cellular Response.

It has been shown previously that the expression of Bcl-2 family members correlated with the cellular response to ABT-737, a highly related Bcl-2 family member inhibitor (See, Tahir, S. K., Yang, X., Anderson, M. G., et al., “Influence of Bcl-2 family members on the cellular response of small-cell lung cancer cell lines to ABT-737,” Cancer Res 2007; 67: 1176-1183). To determine which expression levels of the Bcl-2 family members correlate best to sensitivity to ABT-263, the 14 apoptosis-related genes in the Bcl-2 family that are significantly detected on the microarrays were focused on. Expression values were compared between the sensitive and resistant cell lines, and the results are shown in Table C.

TABLE C
Average Differential Expression of Bcl-2 family members.

| | SCLC | | Leukemia/lymphoma | | Primary Tissue | |
|---|---|---|---|---|---|---|
| | (sensitive vs. resistant) | | (sensitive vs. resistant) | | (SCLC vs. Normal) | |
| Gene | Fold change* | P-value | Fold change | P-value | Fold change | P-value |
| BCL2 (Bcl-2) | −2.1 | 0.021 | −2.7 | 0.026 | −1.5 | 0.02 |
| PMAIP1 (Noxa) | −2.1 | 5.49E−04 | −1.4 | 0.038 | −5.0 | 4.86E−14 |
| BID | | | | | | |
| BCL2L1 (Bcl-x_{L}) | | | | | | |
| BCL2L13 (Bcl-rambo) | | | 1.4 | 5.04E−03 | 1.3 | 0.014 |
| BCL2A1 (A1) | | | 2.9 | 0.039 | 6.3 | 2.53E−06 |
| BCL2L11 (Bim) | | | | | | |
| BAD | | | −1.4 | 0.029 | | |
| BCL2L2 (Bcl-w) | 1.4 | 0.031 | | | 1.2 | 0.013 |
| BBC3 (Puma) | | | | | | |
| BIK | | | | | −6.2 | 4.31E−04 |
| BAX | | | −1.6 | 0.028 | | |
| BAK1 | | | −1.3 | 0.033 | | |
| MCL1 (Mcl-1) | 1.3 | 0.017 | 1.2 | 0.042 | 2.5 | 6.42E−13 |

*Differential expression is shown for all of the resistant cell lines in each panel compared to all of the sensitive cell lines in each panel, p-value is less than 0.05. A negative number indicates the expression in the sensitive cells is higher (by the indicated fold change) compared to the resistant cells.

For the comparison of the entire panel of SCLC cell lines, Bcl-2 and PMAIP1 (or Noxa, an inhibitor of Mcl-1 anti-apoptotic function) are expressed at just over 2-fold higher levels in sensitive cells. Furthermore, Mcl-1 expression is slightly lower (30%) in the sensitive SCLC cells, consistent with the finding that Mcl-1 contributes to resistance to another Bcl-2 family member inhibitor, ABT-737 (See, Tahir, S. K., Yang, X., Anderson, M. G., et al., “Influence of Bcl-2 family members on the cellular response of small-cell lung cancer cell lines to ABT-737,” Cancer Res 2007; 67: 1176-1183). In addition, expression of Bcl-w is slightly lower (40%) in sensitive SCLC cells, although the significance of this was not clear.

Similarly, in sensitive leukemia/lymphoma cells, Bcl-2 and Noxa expression is significantly higher and Mcl-1 is lower (Table C). Expression of Bcl-rambo and the anti-apoptotic gene A1 is also lower in sensitive leukemia/lymphoma cell lines, while expression of the proapoptotic genes BAD, BAK1 and BAX is higher in sensitive leukemia/lymphoma cell lines.

### F. Expression Levels in Primary SCLC Tumors and Normal Lung Tissue.

To compare the expression patterns of the genes between normal and primary tumor tissue, the expression of the Bcl-2 family genes in 8 SCLC tumor tissue samples to 6 normal lung tissue samples taken from the same set of patients was compared. Significantly, it was found that overall, the comparison of the Bcl-2 family genes expressed in primary SCLC tumor tissue to the normal lung tissue paralleled the comparison of sensitive to resistant cell lines. That is, the tumor tissue is similar to the sensitive cells and the normal tissue is similar to the resistant cells in that Bcl-2 and Noxa expression is higher in tumor/sensitive samples while Mcl-1, Bcl-w, A1, and Bcl-rambo expression levels are lower in the tumor/sensitive samples (See, Table C).

### G. SCLC Predictor Gene Sets: Method 1.

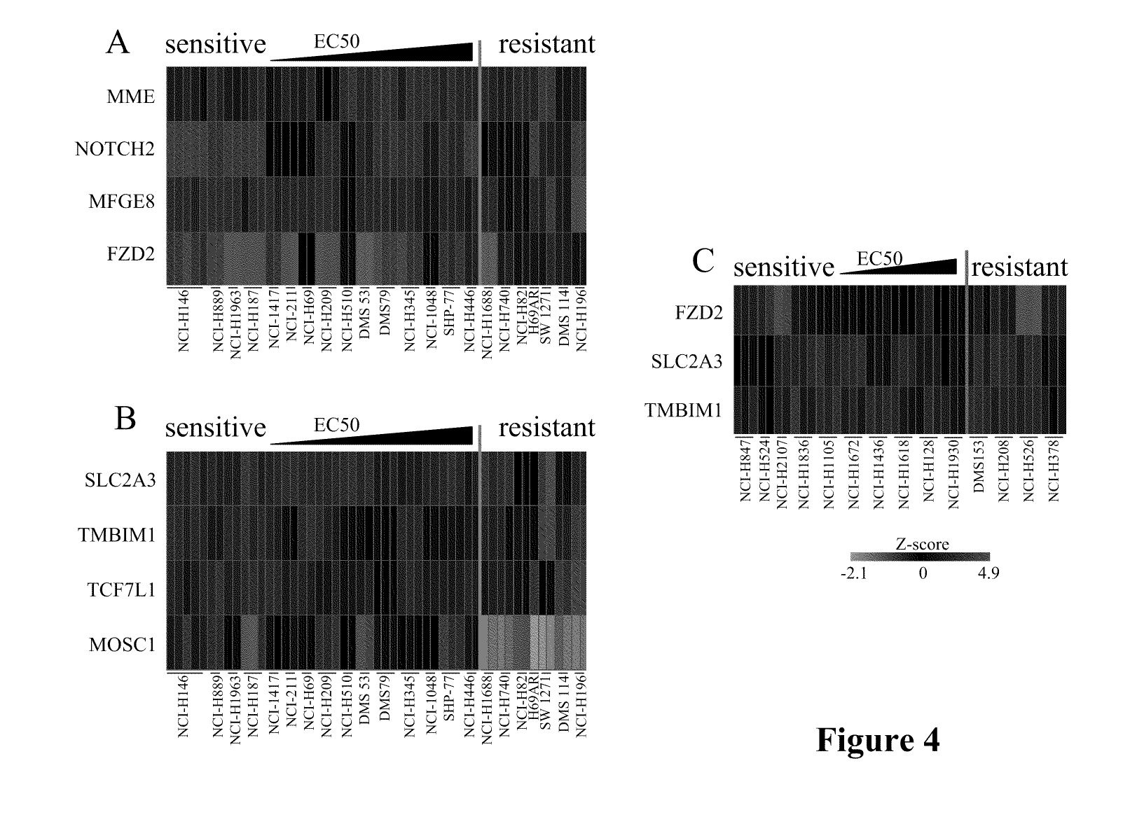

To further interrogate the large-scale data generated with the expression profiles, 2 separate approaches were used to identify predictive sets for sensitivity/resistance to ABT-263. In the first approach, a training set of 15 sensitive and 7 resistant cell lines was created to identify the best sets of markers to predict sensitivity and resistance to ABT-263 using Discriminant Analysis. With this approach, 2 best classifier sets were identified. In a leave-one-out cross validation test (SAS) of these samples with these gene sets, we obtained a 2.0% error rate for set 1, and a 7.8% error rate for set 2, for sensitive versus resistant classification of the sample that was left out. The expression pattern for these genes is shown in FIGS. 4A and 4B. Interestingly, TCF7L1 and FZD2 had higher expression in resistant cell lines and these genes are involved in the Wnt/beta catenin cell signaling pathway. Also expressed higher in the resistant cells was NOTCH2, which is involved in the Notch signaling pathway, MME and SLC2A3, which are cell surface molecules, and TMBIM1, which contains a Bax inhibitor motif, and is part of a family of proteins that may inhibit apoptosis.

A test of these 2 predictor sets was performed on a different panel of SCLC cell lines, comprised of 10 sensitive and 4 resistant lines (See, Table A). Set 1 identified all lines as sensitive, while set 2 classified 62.5% of the arrays for the new lines correctly. Importantly, much higher accuracy (82.6%) was achieved with a classifier of TMBIM1 and SLC2A3 from Set 1 and FZD2 from set 2 (FIG. 4C). To determine the tissue specificity of these sets, the leukemia/lymphoma cell line panel was tested. Set 1 classified 51.3% of the leukemia/lymphoma cell lines correctly, while set 2 classified only 40.8% of the leukemia/lymphoma cell lines correctly. Again, the optimized sets of 3 genes performed better (65.8% of the arrays identified correctly), indicating that this subset of genes is a better predictor for sensitivity.

### H. Leukemia/Lymphoma Predictor Sets: Method 1.

Using the same approach with 17 sensitive and 5 resistant leukemia/lymphoma cell lines, we identified two predictor sets for this panel. In a leave-one-out cross validation test (SAS) of these samples with these gene sets, a 0% error rate was obtained for both sets for sensitive versus resistant classification of the sample that was left out. These sets were distinct from the SCLC predictor sets, and included a cell cycle gene (CCNG1/cyclin G1), and 2 apoptosis genes (BCL2L13/Bcl-rambo and CASP9/Caspase 9) as shown in FIGS. 5A and 5B. To perform a similar forward validation step for these 2 predictor sets, a new panel of 8 sensitive and 1 resistant Mantle Cell Lymphoma cell lines was tested (See, Table B). For each predictor set, 6 sensitive lines were correctly identified as sensitive; however, 1 sensitive line and the one resistant line were incorrectly identified, for an overall accuracy rate of 78%. Significantly, a classifier with CCNG1, PRSS21, and C17orf91 from set 1 and CASP9 from set 2 identified 100% of the arrays from these test lines correctly (FIG. 5C). These sets were tested on the SCLC cell line panel. Leukemia/lymphoma predictor set 1 correctly identified 68.1% of the arrays from SCLC cell lines, while set 2 performed slightly better with 73.6% correctly identified. The optimized set of CCNG1, PRSS21, C17orf91, and CASP9 performed as well as set 2, with 73.6% correct. The predictor sets from the leukemia/lymphoma lines therefore performed better on the SCLC lines than the predictor sets from the SCLC performed on the leukemia/lymphoma cell lines, possibly due to the higher diversity of the cell lines in the leukemia/lymphoma panel.

### I. Generation of Predictor Sets: Method 2.

It should be noted that the estimates of sensitivity and specificity from the leave-one-out method tend to be inflated. So a more rigorous signature selection method was performed where we embedded the entire signature derivation process within a 5-fold stratified cross-validation, and the resulting estimates of sensitivity and specificity should more closely mimic the performance in a future group of similar cell-lines. In this method a simulated annealing algorithm was used within the framework of diagonal linear discriminant analysis (DLDA) and performed 10 replications of 5-fold stratified cross-validation on both the SCLC and the leukemia/lymphoma complete datasets, with each analyzed separately, as described above in Sections 2A-2C. This approach identified a set of 10 genes that predicted 66% of the cell line profiles correctly when evaluated using the rigorous 5-fold stratified cross-validation approach on the SCLC cell lines (See, Table D).

TABLE D
DLDA genes

| Affymetrix ID | Method 1 Genes* | Correct Calls† | Affymetrix ID | Method 1 Genes | Correct Calls |
|---|---|---|---|---|---|
| 202443_x_at | NOTCH2 | | 201828_x_at | FAM127A | |
| 202499_s_at | SLC2A3 | | 205691_at | SYNGR3 | |
| 203435_s_at | MME | | 207180_s_at | HTATIP2 | |
| 210220_at | FZD2 | 82% | 208796_s_at | CCNG1 | 87% |
| 210605_s_at | MFGE8 | | 209116_x_at | HBB | |
| 217730_at | TMBIM1 | | 211560_s_at | ALAS2 | |
| 218865_at | MOSC1 | | 214696_at | C17orf91 | |
| | | | 217650_x_at | ST3GAL2 | |
| Affymetrix ID | Method 2 Genes | Correct Calls | Affymetrix ID | Method 2 Genes | Correct Calls |
| 200872_at | S100A10 | | 201029_s_at | CD99 | |
| 201105_at | LGALS1 | | 201288_at | ARHGDIB | |
| 201231_s_at | ENO1 | | 201310_s_at | C5orf13 | |
| 201477_s_at | RRM1 | | 201347_x_at | GRHPR | |
| 202088_at | SLC39A6 | 66% | 206660_at | IGLL1 | 82% |
| 209366_x_at | CYB5A | | 208892_s_at | DUSP6 | |
| 211528_x_at | HLA-G | | 209806_at | HIST1H2BK | |
| 212063_at | CD44 | | 209942_x_at | MAGEA3 | |
| 216623_x_at | TOX3 | | 211921_x_at | PTMA | |
| 217294_s_at | ENO1 | | 213515_x_at | HBG2 | |
| Affymetrix ID | Bcl-2 family genes | Correct Calls | Affymetrix ID | Bcl-2 family genes | Correct Calls |
| 200797_s_at | MCL1 (Mcl-1) | | 200796_s_at | MCL1 (Mcl-1) | |
| 203684_s_at | BCL2 (Bcl-2) | | 200797_s_at | MCL1 (Mcl-1) | |
| 203685_at | BCL2 (Bcl-2) | 70% | 200798_x_at | MCL1 (Mcl-1) | |
| 204285_s_at | PMAIP1 (Noxa) | | 203684_s_at | BCL2 (Bcl-2) | 81% |
| 204286_s_at | PMAIP1 (Noxa) | | 204493_at | BID | |
| 211725_s_at | BID | | 206665_s_at | BCL2L1 (Bcl-x_{L}) | |
| | | | 209311_at | BCL2L2 (Bcl-w) | |
| | | | 211692_s_at | BBC3 (Puma) | |

*Genes from the SCLC predictor sets (left columns) and leukemia/lymphoma sets (right columns) were combined and processed by Diagonal Linear Discriminant Analysis (DLDA), with the best sets shown.
†The percentage of arrays correctly identified is shown for the sets listed.

The predictor set derived from the leukemia/lymphoma panel using this approach identified 82% of the leukemia/lymphoma cell line profiles correctly. To compare these results to the original predictor sets, the 2 SCLC Method 1 predictor sets were combined, a DLDA was performed, and then tested with 10 repetitions of a 5-fold stratified cross validation. The optimal set of 7 genes identified 82% of the cell line profiles correctly. An identical analysis for the leukemia/lymphoma predictor set identified 8 genes that also performed well, with 87% of the cell line profiles identified correctly. Using the Bcl-2 family genes and applying DLDA with simulated annealing to develop predictive signature sets, the optimal data set included probe sets for Bcl-2, Mcl-1, Noxa, and Bid for SCLC cell lines, with 70% of the SCLC cell line profiles correctly identified. The optimal data set for the leukemia and lymphoma cell lines included probe sets for Bcl-2, Mcl-1, Bid, BCl-X_{L}, Bcl-w, and Puma, with 81% of the cell line profiles correctly identified.

### J. Expression Levels for Signature Sets in Primary SCLC Tumors and Normal Lung Tissue.

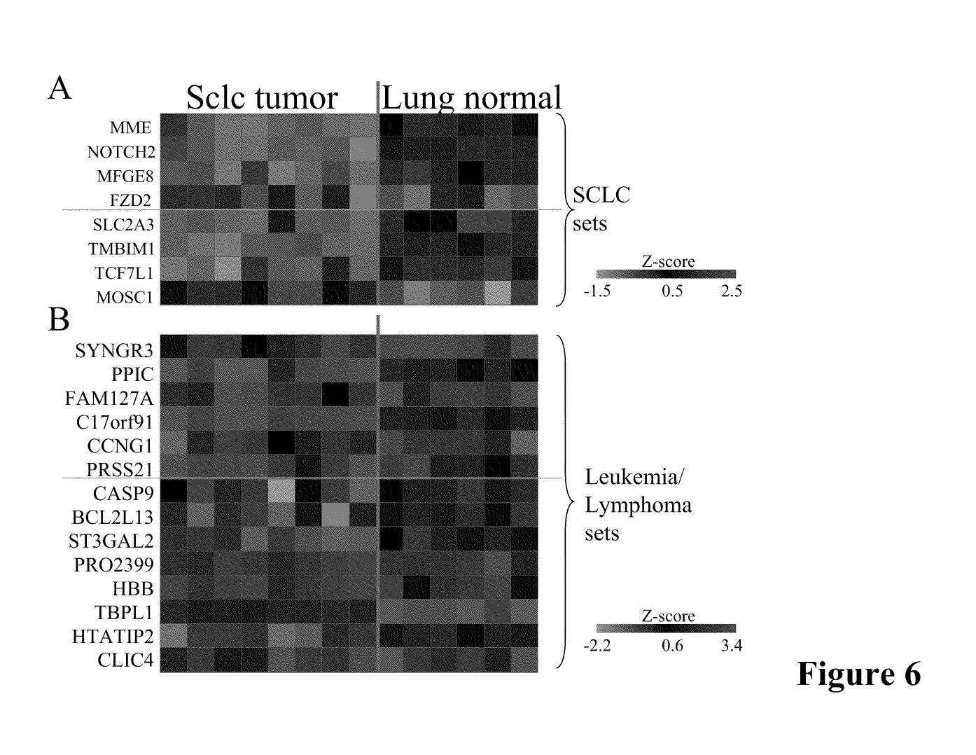

As can be seen qualitatively in a heat map comparison, the expression pattern in SCLC tumor cells for the SCLC predictor sets 1 and 2 is similar to the sensitive cell lines, while the expression pattern in the normal lung tissue is similar to the resistant cell lines (compare FIG. 6A to FIGS. 4A and 4B). As expected, the expression pattern for the leukemia/lymphoma predictor sets does not match the expression pattern seen in either the normal lung or SCLC tumor samples (compare FIG. 6B to FIGS. 5A and 5B). Quantitatively, the SCLC predictor set 1 identified all of the normal tissue as resistant, and 4/8 SCLC tumors as sensitive, while SCLC predictor set 2 also identified all of the normal tissue as resistant, and ⅞ of the SCLC tumors as sensitive.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method of identifying a patient for eligibility for cancer therapy comprising: (a) providing a tissue sample from a patient; (b) determining expression levels in the tissue sample of the biomarker combinations set out in TABLES 1, 2, 3, 4, 5,6, 7, 8, 9, 10, 11, 12, 13, 14, 15, 16, 17, 18, 19 or 20; (c) classifying the levels of expression relative to levels in normal tissue of genes in the corresponding biomarker set; and (d) identifying the patient as eligible to receive a cancer therapy where the patient's sample is classified as having altered levels of genes in the biomarker set.

2. The method of claim 1, wherein the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

3. The method of claim 2, wherein the peripheral blood sample is from a patient with a cancer selected from the group consisting of lung carcinoma and leukemia/lymphoma.

4. The method of claim 2, wherein the tissue sample is a paraffin-embedded fixed tissue sample, a fine needle aspirate or a fresh frozen tissue sample.

5. The method of claim 1, wherein the determining step (b) is performed by in situ hybridization.

6. The method of claim 5, wherein the in situ hybridization is performed with a nucleic acid probe that is fluorescently labeled.

7. The method of claim 5, wherein the in situ hybridization is performed with at least two nucleic acid probes.

8. The method of claim 5, wherein the in situ hybridization is performed with a peptide nucleic acid probe.

9. The method of claim 1, wherein the determining step (b) is performed by polymerase chain reaction.

10. The method of claim 1, wherein the determining step (b) is performed by a nucleic acid microarray assay.

11. The method of claim 1, wherein the patient is classified as eligible to receive an anti-sense therapy compound designed to bind to one of Bcl-2, Bcl-w, and Bcl-xl.

12. The method of claim 1, wherein the cancer therapy comprises a Bcl-2 family inhibitor.

13. The method of claims 11 or 12, wherein the patient is classified as eligible to receive N-(4-=(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

14. The method of claim 11 or 12, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

15. The method of claim 1, wherein the cancer therapy comprises a Bcl-2 family inhibitor in combination with chemotherapy.

16. The method of claim 15, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

17. The method of claim 15, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

18. A method of identifying a patient for eligibility for Bcl-2 family inhibitor therapy comprising: (a) providing a lung cancer tissue sample from a patient; (b) detecting the level of expression in the tissue sample; wherein differential expression of the biomarker combinations set out in TABLES 1, 2, 3, 4, 5 or 6 is indicative of a patient being eligible to receive Bcl-2 family inhibitor therapy.

19. The method of claim 18, wherein the determining step (b) is performed by PCR.

20. The method of claim 18, wherein the determining step (b) is performed by a nucleic acid microarray assay.

21. The method of claim 18, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

22. The method of claim 18, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

23. The method of claim 18, wherein the patient is classified as eligible to receive an anti-sense therapy compound designed to bind to one of Bcl-2, Bcl-w, and Bcl-xl.

24. The method of claim 18, wherein the cancer therapy comprises a Bcl-2 family inhibitor in combination with chemotherapy.

25. The method of claim 24, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

26. The method of claim 24, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

27. A method of identifying a patient for eligibility for Bcl-2 family inhibitor therapy comprising: (a) providing a leukemia/lymphoma tissue sample from a patient; (b) determining expression levels in the tissue sample of the biomarker combinations set out in TABLES 7, 8, 9, 10, 11, 12, 13, 14, 15, 16, 17, 18, 19 or 20; (c) classifying the level relative to levels in normal tissue of genes in the biomarker set; and (d) identifying the patient as eligible to receive Bcl-2 family inhibitor therapy where the patient's sample is classified as having a altered levels of genes in the biomarker set.

28. The method of claim 27, wherein the determining step (b) is performed by PCR.

29. The method of claim 27, wherein the determining step (b) is performed by a nucleic acid microarray assay.

30. The method of claim 27, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

31. The method of claim 27, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

32. The method of claim 27, wherein the patient is classified as eligible to receive an anti-sense therapy compound designed to bind to one of Bcl-2, Bcl-w, and Bcl-xl.

33. The method of claim 27, wherein the cancer therapy comprises a Bcl-2 family inhibitor in combination with chemotherapy.

34. The method of claim 33, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

35. The method of claim 33, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

36. A method for monitoring a patient being treated with Bcl-2 family inhibitor therapy comprising: (a) providing a peripheral blood sample from a patient; (b) measuring expression levels in the peripheral blood sample of the biomarker combinations set out in TABLES 1, 2, 3, 4, 5, 6, 7, 8, 9, 10, 11, 12, 13, 14, 15, 16, 17, 18, 19 or 20; and (c) determining the expression level relative to a patient baseline blood level of the biomarker combinations set out in TABLES 1, 2, 3, 4, 5, 6, 7, 8, 9, 10, 11, 12, 13, 14, 15, 16, 17, 18, 19 or 20.

37. The method of claim 36, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

38. The method of claim 36, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

39. A computer system comprising: (a) a database containing information identifying the expression level in lung cancer tissue of a set of genes set out in Table 1, 2, 3, 4, 5, 6, 7, 8, 9, 10, 11, 12, 13, 14, 15, 16, 17, 18, 19or 20;and(b)a user interface to view the information.

40. A computer system of claim 39, wherein the database further comprises sequence information for the genes.

41. A computer system of claim 39, wherein the database further comprises information identifying the expression level for the genes in normal tissue.

42. A computer system of claim 39, wherein the database further comprises information identifying the expression level for the genes in tissue from a lung tumor.

43. A computer system of any of claims 39-42, further comprising records including descriptive information from an external database, which information correlates said genes to records in the external database.

44. A computer system of claim 43, wherein the external database is GenBank.

45. A method of using a computer system of any one of claims 39-42 to present information identifying the expression level in a tissue or cell of the biomarker combinations set out in TABLES 1, 2, 3, 4, 5, 6, 7, 8, 9, 10, 11, 12, 13, 14, 15, 16, 17, 18, 19 or 20 comprising: (a) comparing the expression level of the biomarker combinations set out in TABLES 1, 2, 3, 4, 5, 6, 7, 8, 9, 10, 11, 12, 13, 14, 15, 16, 17, 18, 19 or 20 in the tissue or cell to the level of expression of the gene in the database.
